SEQUENCE LISTING (21 sequences)

SEQ ID NO: 1 - DNA, 7554 bp, Homo sapiens
ccaatgatatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagacaacaccacggaattatcagtgcccagcaacctagcccctgtccagcagcgggcaaggcaggcggcgatgagttctgccgtggcgatcgggagggggaaagcgaaagtcccagaaaggagttgacaggtggtggcaatgccccagccagtgggggttgcgtcagcaaacacagagcacaccacgccacgttgacggacaacgggccacaactcctctaaaagagacagcaaccaggatttatacaaggaggagaaaacgaaagccgtacgggaagcaatagctagatacagaggctataaagcagcatatccacacagcgtaaaaggagcaacatagttaagaatatcagtcaatctttcacaaattttgtaatccagaggttgattaacaggaacagagcgtaaataacgggaaagtttcttaacatgtttgtcttgtggcaatacacctgaactagtaattacatatccctaaaaatgtaaatgattgccccaccattttgttttattaacatttaaatgtatacccaaatcaagaaaaacagaacaaatatgggaataaatggcggtaagatgctccgccggcgtcacttgcggaacctgctcatctcgatgtcgttatagatccgcttattgttagagccctgatatctgtaccgcacggcgatcacgatccttgccaccacgtacagcaggatgaacacgccgatggcgaaaaagaaggtcaccactgtgctcttccaggagctaaaccagccctcgatcagctccacgggattcttagagatgccggtatcgccaaagaacagtgtctcctcctcaggcagctgctttggtgcctctgccaggtgtgggtgctcaaacacctcggcctgggaggtcttgtgcagatcgctgtccagcatgccgtggccgatcatgaacaggggaaacttatagccggttggtgtcttcaggatgccgtttgggccgatctccacgccctcgtaagggaaccactcggtccacagctctctctctgtctgagagccggagatcttgcccaccatcttgctgatgataggattatcgatgtcgattctgatataccgggtctcgaagtacttcagggtgccgttgatgattgtaaaggctggtccggttcctggattctttggtgccagatagctcaggtccacaggagacacgggctgcttgctcctgatcttagaccatgtctcctggcacaggctgtagtccaggattctctccacatccaggatcagggacacgtccacgctggtctgtgtaggggcggagatggttgctcccactgggcactcaggcagctttgctgctgcgtacacgtcctggtccacgaactcaaaccacacgccgcttggcagccgcaccccggcgtgcttacaatagttcatcttgcacaccttatcgcccttctcataggcgaagtagttggagcggtagccggtattgggcttgccgatgctctccttcttgccgtcctcagaaaagaaggtgatctctgtatccaccagggtggcgtcgcacaggcctgtcaccttataatcggagtaccacactgtgctgttgtgcacggtctcacactcctctgtctcgcacttgccatttggaaactggctgtcgatccactcgcctgtatactcatccaccagcacgtggtgaggggttgcctgcaccaccactgccacggagtcggtcactgtggcgtagccacaattctgaggtgggaatcctgggctcatccaggttccctgctttgtctgcttgatagactccttgcactgctcggatgtaggctggatggagtggatgctgtgggtgatatacttggggccgtaccaccgaaagtcacaggttgtgatccacttggctgcgtggcacatccatccatctgcctggatggccttgtgggtctttggcatcttcaccttcattgtgatgcccagcagatcattgtgccagttctggtcagaggagctagggcaatagtggtaagaggagggcacattcttccagttgcccttctggctctgtgggaacacgatagaaaacttggcgtgggagcacagaggcagcacgatgaatgtcagcagcagaaagttggggcctggattttcttccacgtcgccacaagtcagcagggagcctctgccttcgccagagccctcggacctagggcttggcaggccggcagggatctctggtgtcactctgaacaggcccagcacggttgctccctgtgtcagctgccatgcgtggcgggcccttgcctcggtgtgcaggtgcactcccagcctctgtccggcggacaggtgcagcagtctgccctggaagccaaaggcgctgttccgtgcctcgctagaggctggaggcagatccacggtcagggccagggcggcggccccggcggcgctccgcagtggctgcaggtgcagggccaggctcacagagccggagccctcgccggccaccactctcctcagctccagctggaagaacacgtagtacacgccggccttggccaccaccagctcctttgtgtcctccttatagctcagtcctccggtcagagacacgccggccaggcctgggtcagagtaccaggacagagggccatcgatcagcagcacattctgggccaccagctgggcaaacattccctgcctcagatccagcaggccggcaggatcgtctgggctcagctcaggtccctctctcagccttgggctggcggcagagcctggggaggctctggcgccggacacggcccagggacaggccaggaacacggcgcaggcggcggccagcagcagcagtaataacaggccggccaccagggcccaaggcagcacccggcaggcccttgcccttggggcgggtggccatggggcctcggggtccaggctggcgtcagaggcgtactccattgggccagggttctcctccacgtcgccacaggtcagcagggagccccttccctctccgctgcccttctttctgccccgcttgcaatacagggtgatgaccaggctcagcagcagcacgccacaggttccggccagaggtgcccagatgtagatatcgcaggcaaagtccagtcccctggtgtgcactgccccccctgcggcagggcgacaggcctcgggcctcagagacagtggctgggaggcgatggttggggctggtgtaggtggcctaggggcgggggttgtggtagaggggtcgcgcttgatctccagcttggtgccgctgccgaaggtgaaggggttggagctccactgctggcaatagtaggttgctgcatcctctgcctccattccgctgatggtcaggctataagatgtgccggagccgctgcccctgaagtgtgcaggcactccgctggccagcttagatgtgtcgtagatccatctcttgggggaggtgccgctcttctgctgataccaattcatgtagctcacgctggagctggcggagcaggtcattgtcaccttctctcctggagaggcggacatgatggctggggactgggtcatcacgatatcgctgccgccgccgccagagcctcctccgccggagcctcctcctccagaggacacggtcaccagtgtgccctggccccaatagtccagacagtagtgatcgtcatagtagccggcgcaatagtacacggcagaatcctcggatgtcaggctagacagctgcatgtaggcggtggagctagacttgtctgtggtcagtgtggccttatccttgaacttctgattatagtttgtgtagccgcggctggggttgatgtagccgatccactccagtccctgtcctggcctctgcttcacccagtgcatggtatacctggtgaaggtgtagccggaggccttacagctcatcttcacagaggctcctggccgggccagctctgctccggactccagcagctgcacctgagggcgggcggcgtgcagcagcagggccagaggcagcagcagggctgtcactggcagggctggtccaggattctcctccacgtcgccgcaggtcagcaggctgccccttccctctccagagccgaaacaggtatcggacttgtcgcaggagcttgtcttagaggacttaaacactctctgggcctcatcgctccgctctgggatgtggatcttctctctcttcttggtctgctcagactcctcccgctccatggtgtttgtgccacacttgtaggaattgcgaggccgcttcttcttcttccacttccacaggatcagacagaacaccatcacgcagatgatcacggttggcagcacggctgtgatccaagggatgtgatctggaggtggctgggggtcctccagctcgatagaaaatgggctagacagcaggcgggtcttgtctgtctccaggatgcagaagatggtcatgttggatgtcacatctggaaagctcacagacagggagatgctcacgtcatacagctcggtcacgttatcctgggacttctgcatgatgccgtcgtactcgatggtgctgttctttgtcctcagcagcacgctcatcttcttgggctctggatagccgtggatggagctacaggtcaggttgatgtacacgttctctgtgatgttagagattggcacgatctcaggctgggaaaagttggccagcacgctcagctcgctgttcatctggtggattctgatcatgccggtaggcttcttgtggtggatgatgcactggtacaggcccttatccttgatctgcagattgtgcagcctcagggtccaagaatcggagtcgaagctggtccggcccatatacttggagtgcacgctgtcaaacttctccttgcccaggtacacctcattcagcaccaggttctcctgatcctgccagaacaccaccagctcgctcagagactgattctgggagttggcgaactgacagggcaggtcggctgtctcgttaaagtaggcctggatcttcagtggtgctgctccgctcagcaggaaggccatcacaaacaggatattagacaggcccatggtgcactgggggtccatggtggcggccctgcaggcttctatggaagtcaaaacagcgtggatggcgtctccaggcgatctgacggttcactaaacgagctctgcttatatagagctcggggagcagaagcgcgcgaacagaagcgagaagcgaactgattggttagttcaaataaggcacagggtcatttcaggtccttggggcaccctggaaacatctgatggttctctagaaactgctgagggcgggaccgcatctggggaccatctgttcttggccctgagccggggcaggaactgcttaccacagatatcctgtttggcccatattctgctgttccaactgttcttggccctgagccggggcaggaactgcttaccacagatatcctgtttggcccatattctcctgtttctctgttcctagacgtc

SEQ ID NO: 2 - DNA, 7653 bp, Mus musculus
ccaatgatatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagacaacaccacggaattatcagtgcccagcaacctagcccctgtccagcagcgggcaaggcaggcggcgatgagttctgccgtggcgatcgggagggggaaagcgaaagtcccagaaaggagttgacaggtggtggcaatgccccagccagtgggggttgcgtcagcaaacacagagcacaccacgccacgttgacggacaacgggccacaactcctctaaaagagacagcaaccaggatttatacaaggaggagaaaacgaaagccgtacgggaagcaatagctagatacagaggctataaagcagcatatccacacagcgtaaaaggagcaacatagttaagaatatcagtcaatctttcacaaattttgtaatccagaggttgattaacaggaacagagcgtaaataacgggaaagtttcttaacatgtttgtcttgtggcaatacacctgaactagtaattacatatccctaaaaatgtaaatgattgccccaccattttgttttattaacatttaaatgtatacccaaatcaagaaaaacagaacaaatatgggaataaatggcggtaagatgctccgccggcgtcacttgcggaacctggacatctcgatatcgttatagattctcttattgttgctgccctgatatctgtaccgcacggcgatcacgatccttgccaccacgtacagcaggatgaacacgccgatggcgaaaaagaatgtcaccacggtgctcttccagctagaaaaccagccctcgatcagctccacggggttcttagagatgcctgtgtcgccaaagaacagggtctcctcctctggcagctgctttggtgcctctgccaggtgtgggtgctcaaacacctcggcctggctggtcttgtgcagatcagagtccagcatgccgtggccgatcatgaacaggggaaacttatagcctgttggggtcttcaggatgccattagggccgatctccacgccctcgtaggggaaccactctgtccacagctctctctcggtctgagagccggagatcttgcccaccatcttgctgatgatagggttatcgatgtcgattctgatataccgggtctcgaagtacttcagtgtgccgttgatgatggtaaaggctggtcctgttcctggattctttggtgccagataagacagatccacaggggacacgggctgcttgctgcggatcttagaccaggtctcctggcacaggctgtaatccaggattctctccacgtccaggatcaggctcacgtccacgcttgtctgggttggggcagagattgtggcgcccactgggcactcaggcagctttgctgctgcgtacacgtcctggtccacgaactcaaaccacacgccgctaggcagccgcaccccggcgtgcttacaataattcatcttgcacaccttgtcgcccttctcataggcgaagtagttggacctgtagccggtattgggcttgccgatggactccttcttgccatcctcgctaaagaatgtgatctcggtgtccaccagtgtggcatcgcacaggccggtcaccttatagtcgctgtaccacacggtagaattgtgcactgtctcacactcctcggtctcgcacttgccgttgggaaactggctatcgatccactcgccggtatactcgtccaccagcacgtggtgtggtgttgcctgcaccaccactgccacggagtctgtcacggtggcgtagccacagttctgaggtgggaatcctgggctcatccatgttccctgcttggtctgcttgatggactccttgcactgctcgctggttggctggatagagtggatggagtgtgtgatatacttagggccgtaccagcgaaaatcacaggttgtgatccacttggctgcgtggcacatccatccgtctgcctggatggccttgtgtgtctttggcatcttcaccttcatggtgatgcccagcaggtcattgtgccagttctgatcggagctagatggacaatagtggtaggagctaggcacattcttccagttgcccttctgggactgggggaacacgatggaaaacttggcgtggctgcacagtggcagcacgatgaaggtcagcagcagaaagttagggccgggattctcctccacatcgccacaggtcagcagagagccccttccctcgccggagccttcccatggattgtctggcttcaccaggaacaggccaaagctggttgtgttgggataagacagctcccagtctctgtaggcatcctgggcgccgtgcaggtaggcgcgcagtcccacgctcagcctgtgtcctgccttcagcagcagcagctggctccaagaccgatccaccagcttattctccatggagcatgggaacagctccacggtcagggccaggttgtcaaaatcgtccacctgaggcttggcctgcagcaccaggctcacccatccctgcaccttgtggcctgtattggtgaatgtaggagacagcttcagctccaggaacacgtagtacaggccgggggaatccaccaccagctccttcttgtcctcctcataccgcaggccctgagacaggtaagaggagcctgctccatcctgggagtgccagttcagggttgtattgcacaggctggcctgattcttggccagcagcttggcgaacactggagagccctgctgggttgtgttagggcagccgatgtgggacactggggtcacctggtcggcattgttctcgcgtgtgcccaggttaggggaggttgtgatggtcagggctgggcgaggctcggtccttgtgaagatgggcacgcatgctgcgatcagcagcagcagcaccagggccaccagtccatacagcttagggtgccggctgcaaaaattcagggctggaggccatgctgcctccctatccctcacgttcactgcagggtatgctgcgtcggttggcagggcggcatttgttgggcgcacggtgtcggacagcagggcggcatcggccagcagtccggtgtctctcagcagggcggcatcgctagggcaagatgtgccggcggggtgccttgcgtctgctgtatcctccacgtccagggtgtgctgatccatggggcctgggttctcctccacgtcgccacaggtcagcagggagccccttccctctccgctgcccttctttctgccccgcttgcaatacagggtgatgaccaggctcagcagcagcacgccacatgttccggccaggggtgcccagatgtagatgtcgcaggcaaaatccagtcccctggtgtgcactgccccccctgcggctgggcgacaggcctcaggcctcagagacagtggctgggaggcgattgtaggggcgggggttggtggccttggggcaggggttgtggtgctggggtcccgcttgatctccagctttgtgcctgggccgaaggtccaagggtagttatagtactgctggcaatagtaagagccgatatcctcggactccaggctagagatggtgaaggagctgtctctgccggagccgctgccagaaaaccgggatggcactccgtcggccagcttatttgtatagtagatcagcagcttaggggccttgccgggcttctgctgataccaattcaggtagttgctgatatcctgagaggcctgacagttgatggtcaccctgtcgcccagagaggctggcagagaggaaggggactgtgtcatctggatatcgctgccgccgccgccagagcctcctcctccggagcctcctcctccgctagacactgtcaccatggtgccctggccccagtaattcttatcccagtcgaatctggcgcaatagtacatggcggtgtcctcggacttcaggatgttcatctgcaggaacagcagattcttggcgttatctctgctcacggtaaaccggcccttcacggcgtcggcatacttgatgttgatggagctagatgtgatgtaggccacagactccagtccccttcctggtgcctgcctcacccagtgcatgccgtagccggagaaggtaaagccggaggcctcacagctcagcttcagagacttgccaggctgcaccagtcctcctccgctctccaccagctgcacctctggcctggcggcgtgcagcagcagggccagaggcagcagcagggcggtcactggcagggcgggtcctggattctcctccacgtcgccgcatgtcagcagagagcctcttccctctccggagccctctgcgtttggcttggcggaggcgatctgaggctccagctccttcaggttgatggtctctctgtcggcattgctatcccgctccagcttagaggctgtgttggaaggccggctgggctgatttggcttcttgtggcacacgatgatcagcagcatgaccagcagcagggccactgtcacgctggcggtgatctccttccagtatgtctgaggagaggggaactcctgggtgaagttcaggggcttggagctgatcttcatgctctctgtctccagcacgcacaccacggtcatgtgccacacgccgtcagggaaggacaggctcagagagttggagatgctaaacagctcggtcacgttatcctgggagatctgcatattgtcgccatactcatttgtgctgttggtgatcaggaagtacatcttctttggcttagggtggccctgcttgctggtacatgtcaggttgatgccagaattgccggtcacgttctgggccagcttgatctcgggctcggaaaagttggcgatcacgctcagctcggtcagtgtctgctgcaggatgatgctgccggtagggggcttcttctggatgaagcagtcataagagcccatatccttgatctgcacattgtgcagcctcagtgtccaattgtttctatcaaaagaggtccggcccaggtacttggcgttcacggagtccagcttctcggtgcccagatagtgctcgtacagcaccagcttctgctggtcctgccaaaacaccaccagctcagacagggagatattctgggcctttgtgaagggacatggcagataggctgtgccgttaaagtaggcctgggtctccacggacacggcatcgctgatcagcagcacggtcacgaagatcaggatggccaggcccattgtgcatctagggtccatggtggcggccctgcaggcttctatggaagtcaaaacagcgtggatggcgtctccaggcgatctgacggttcactaaacgagctctgcttatatagagctcggggagcagaagcgcgcgaacagaagcgagaagcgaactgattggttagttcaaataaggcacagggtcatttcaggtccttggggcaccctggaaacatctgatggttctctagaaactgctgagggcgggaccgcatctggggaccatctgttcttggccctgagccggggcaggaactgcttaccacagatatcctgtttggcccatattctgctgttccaactgttcttggccctgagccggggcaggaactgcttaccacagatatcctgtttggcccatattctcctgtttctctgttcctagacgtc

SEQ ID NO: 3 - DNA, 7584 bp, Canis familiaris
ccaatgatatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagacaacaccacggaattatcagtgcccagcaacctagcccctgtccagcagcgggcaaggcaggcggcgatgagttctgccgtggcgatcgggagggggaaagcgaaagtcccagaaaggagttgacaggtggtggcaatgccccagccagtgggggttgcgtcagcaaacacagagcacaccacgccacgttgacggacaacgggccacaactcctctaaaagagacagcaaccaggatttatacaaggaggagaaaacgaaagccgtacgggaagcaatagctagatacagaggctataaagcagcatatccacacagcgtaaaaggagcaacatagttaagaatatcagtcaatctttcacaaattttgtaatccagaggttgattaacaggaacagagcgtaaataacgggaaagtttcttaacatgtttgtcttgtggcaatacacctgaactagtaattacatatccctaaaaatgtaaatgattgccccaccattttgttttattaacatttaaatgtatacccaaatcaagaaaaacagaacaaatatgggaataaatggcggtaagatgctccgccggcgtcacttcctgaatctgctcatctcgatgtcattatagatcctcttattgttagagccctgataccggtagcgcacggcgatcacgattctggccaccacgtacagcaggatgaacacgccgatggcgaaaaagaatgtcaccacggtagacttccaggagctaaaccagccctcgatcagctccacagggttcttggagatgcctgtatcgccaaagaacagggtctcctcctcaggcagctgctttggtgcctctgccaggtgtgggtgctcaaacacctcggcctggctggtcttgtgcagatcagagtccagcatgccgtggccgatcatgaacagtggaaacttatagcctgtaggggtcttcaggatgccattagggccgatctccacgccctcgtaggggaaccactctgtccacagctctctctcggtctgggagccgctgatcttgcccaccatcttgctgatgattgggttatcgatgtcgatccggatatagcgggtctcgaagtacttcagtgtgccattgatgatggtaaaggcaggtcctgttcctgggttctttggtgccagatagctcaggtccacaggagacacgggctgcttggaccggatcttgctccaggtctcctggcacaggctgtagtccaggatgcgctccacatccaggatcagagacacgtccacggatgtctgggtgggggcggagatggttgctcccactgggcactcaggcagctttgctgctgcgtacacgtcctggtccacgaactcaaaccacacgccgcttggcagccgcaccccggcgtgcttacaataattcatcttgcacaccttatcgcccttctcataggcgaagtaattggatctgtagccggtgttgggcttgccgatagactccttcttgccgtcctcggaaaagaatgtgatctcggtatccaccagtgtggcgtcgcacaggccggtcaccttataatcggagtaccacacggtgctattgtgcactgtctcacactcctcggtctcgcacttgccgttgggaaactggctgtcgatccactcgcctgtatactcatccaccagcacgtggtgaggggttgcctgcaccaccactgccacgctgtctgtcacggtggcgtagccacagttctgtggagggaagcctggagacatccatgtgccctgcttggtctgcttgatggactccttgcactgctcgcttgtaggctggatgctgtggatagagtgggtgatatacttggggccgtaccacctaaagtcacaggttgtgatccacttggctgcgtggcacatccatccatctgcctggatggccttgtgtgtcttaggcatcttcaccttcatggtgatgcccagcagatcattgtgccagttctggtcagaggagcttgggcaatagtggtaagaggaaggcacattcttccagttgcccttctggctctgtgggaacacgatagaaaacttggcgtgggagcacagaggcagcacgatgaatgtcagcagcagaaaatttggtccagggttctcctccacgtcgccgcaggtcagcagagagcctctgccctcgccggagccctctccgtccagtggtggggagcctggtccggtatccattggccagctagaaggcagtcctgttggcaccttggttgccacccggaacagtcccaggattgttgctccctgggccagctgccatgcagggtgtgctcctgcctctgccctcaggtgcaccctcagtctctgtcctgcgtccaggtgcagcaggctgcccctaaagcctgcggcagaatctctggcctcggagctagggggtggcagatccagtgtcaggtccagggcggctgcctcggttcccagtggctgcaggtgcagggcggcagacacggagccgctgccggtagaggacatcactctctgcagcttcaggtgcaggaacacgtagtacaggccaggctccaccaccatcagctctcttgtgtgctggtcataggacaggccgggtcccagaaacacgccggccaggccggggtcgctgtaccacctcagtggtccctccttcagctgcacatctctggccaccagctgtgcgaacacggcctgtgggctgtctggcagcctggcgccggcatcgggcagtggggcaggcagggcgggcagggcagggggccttggtcctggcaccacccaggcgcgcagggcacaggcggcgcaggtgcccaccagcagcagcattgcggcggacagtgcccaaggcagtgggctgcaggctctgcctggaggggcgggtggccttggtgcctctggatcaggggcggcgtcgctccgtggcctcattggtcctgggttttcttccacatcgccgcaagtcagcaggctgccccttccctctccagagcccaccacaggtcttggacacttgcacacccggcgcctattccggtggttgcaatacagggtgatgaccaggctcagcagcagcacgccacaggttccggccaggggtgcccagatgtagatatcgcaggcgaagtccaggccgcgggtgtgcactgccccccctgcggctggcctacaggcctctggtctcaggctcagaggctgagaggcgattgttggggcaggggtaggtggccttggggctggggttgtggtggggtcgctccgcttgatctccaggtttgtgccagagccgaaggtaaaaggataggagtaggtctgtccgcagtggtatgctgccagatcctcggactgcacgcttgtgatggtcagtgtgaagtctgtggcagagccggagccggtaaacctatcgggcacgccggtagatctattggaggcgccgaagatcagcagctgtgggctctggtcaggcttctgctggtaccagctcacaaaagaatccacgttctcggaggcccgacagctcagtgtcaccctctctcccacgctcatagacatggactttggggactgggtcatcacgatgatgctgccgccgccgccagagcctcctcctccggagcctcctcctccgctagaggacacggtcactgtggttcctgctccccacacgtcgaaatagcctctccgcacgcaatagtacatggctgtatcctcggtcttcagattgttcatctgcaggtacagcatgctctgagaatcgtcccgggagattgtaaagcggtccttcacgctgccggcatagtaggtggcataattcttggacttgctcctgattctggccacccactccagtcccttgcctggtgcctgtctcacccaattcatggcgtagatgttgaatgtaaagccagaggtggcgcaggacagcttcaggctgcccttgggctgcaccagtcctcctccgctctccaccagctgcacctctggcctggcggcgtgcagcagcagggccagaggcagcagcagggctgtcactggcagggcaggtcctgggttctcctccacatcgccacaggtcagcagggagcctcttccctctccgctgccgaactgtgtggtagaattgtcgccggaggcggtcttgctgatgttcacgcactgggcctcatcggaccgctctgtctcgtggtaccgcacgcgctccttggtctgctcagactccttgcgctccaccttatttgtctcacactcgtgggatgggccaggctgcttcttcttccgctttctcagggtcagaaagaacaccatgccgcacaggatgaccagcatgaccagcagggcggcgatccacaggatgtgatcgccgtcgggtgttggcttggtgtgggcgtcgatgttgtaaggcaggctgggcagcttcatagactccagctgcagcacgcagaagatgctcacgttgctggcctcaggcacagaaaaggacaggctgatagacacgttatacagctcggtcacattgttctggctcttcttcatcactgtatcgtacttggtgctgctgttctctgtcttcaccaggaaatacatctccttaggctcggggtagccctggatggagctacaggtcaggttgatgatgccgctgttctctgtccggttggaggtcaccatgatctcgggctggctgaagttggccagcacagacaggtcggagttcatctggtgcatgggcaccaggccctttgggcccttgtggtgcacgaagcactgatacaggcccttatccttgatctggatattgtgcagccgcagtgtccagttatccttgtcaaaggaggtcctgcccttgtactttctgtgcacattctgagggttctccttgccgcgatacagctcgtacagcaccagcttatcctggtcctgccaaaacaccaccagctcgtccagagagatattctgggagtttgtgaagtgacagggcagctcgccggtcttgttgaagtaggcctggctcttcatagaggcggcgccgtacagcagcagtgtcatcacgaacaggatattgttcagctccatggtgcagcgcaggtacatggtggcggccctgcaggcttctatggaagtcaaaacagcgtggatggcgtctccaggcgatctgacggttcactaaacgagctctgcttatatagagctcggggagcagaagcgcgcgaacagaagcgagaagcgaactgattggttagttcaaataaggcacagggtcatttcaggtccttggggcaccctggaaacatctgatggttctctagaaactgctgagggcgggaccgcatctggggaccatctgttcttggccctgagccggggcaggaactgcttaccacagatatcctgtttggcccatattctgctgttccaactgttcttggccctgagccggggcaggaactgcttaccacagatatcctgtttggcccatattctcctgtttctctgttcctagacgtc

SEQ ID NO: 4 - DNA, 4491 bp, Homo sapiens
atggacccccagtgcaccatgggcctgtctaatatcctgtttgtgatggccttcctgctgagcggagcagcaccactgaagatccaggcctactttaacgagacagccgacctgccctgtcagttcgccaactcccagaatcagtctctgagcgagctggtggtgttctggcaggatcaggagaacctggtgctgaatgaggtgtacctgggcaaggagaagtttgacagcgtgcactccaagtatatgggccggaccagcttcgactccgattcttggaccctgaggctgcacaatctgcagatcaaggataagggcctgtaccagtgcatcatccaccacaagaagcctaccggcatgatcagaatccaccagatgaacagcgagctgagcgtgctggccaacttttcccagcctgagatcgtgccaatctctaacatcacagagaacgtgtacatcaacctgacctgtagctccatccacggctatccagagcccaagaagatgagcgtgctgctgaggacaaagaacagcaccatcgagtacgacggcatcatgcagaagtcccaggataacgtgaccgagctgtatgacgtgagcatctccctgtctgtgagctttccagatgtgacatccaacatgaccatcttctgcatcctggagacagacaagacccgcctgctgtctagcccattttctatcgagctggaggacccccagccacctccagatcacatcccttggatcacagccgtgctgccaaccgtgatcatctgcgtgatggtgttctgtctgatcctgtggaagtggaagaagaagaagcggcctcgcaattcctacaagtgtggcacaaacaccatggagcgggaggagtctgagcagaccaagaagagagagaagatccacatcccagagcggagcgatgaggcccagagagtgtttaagtcctctaagacaagctcctgcgacaagtccgatacctgtttcggctctggagagggaaggggcagcctgctgacctgcggcgacgtggaggagaatcctggaccagccctgccagtgacagccctgctgctgcctctggccctgctgctgcacgccgcccgccctcaggtgcagctgctggagtccggagcagagctggcccggccaggagcctctgtgaagatgagctgtaaggcctccggctacaccttcaccaggtataccatgcactgggtgaagcagaggccaggacagggactggagtggatcggctacatcaaccccagccgcggctacacaaactataatcagaagttcaaggataaggccacactgaccacagacaagtctagctccaccgcctacatgcagctgtctagcctgacatccgaggattctgccgtgtactattgcgccggctactatgacgatcactactgtctggactattggggccagggcacactggtgaccgtgtcctctggaggaggaggctccggcggaggaggctctggcggcggcggcagcgatatcgtgatgacccagtccccagccatcatgtccgcctctccaggagagaaggtgacaatgacctgctccgccagctccagcgtgagctacatgaattggtatcagcagaagagcggcacctcccccaagagatggatctacgacacatctaagctggccagcggagtgcctgcacacttcaggggcagcggctccggcacatcttatagcctgaccatcagcggaatggaggcagaggatgcagcaacctactattgccagcagtggagctccaaccccttcaccttcggcagcggcaccaagctggagatcaagcgcgacccctctaccacaacccccgcccctaggccacctacaccagccccaaccatcgcctcccagccactgtctctgaggcccgaggcctgtcgccctgccgcagggggggcagtgcacaccaggggactggactttgcctgcgatatctacatctgggcacctctggccggaacctgtggcgtgctgctgctgagcctggtcatcaccctgtattgcaagcggggcagaaagaagggcagcggagagggaaggggctccctgctgacctgtggcgacgtggaggagaaccctggcccaatggagtacgcctctgacgccagcctggaccccgaggccccatggccacccgccccaagggcaagggcctgccgggtgctgccttgggccctggtggccggcctgttattactgctgctgctggccgccgcctgcgccgtgttcctggcctgtccctgggccgtgtccggcgccagagcctccccaggctctgccgccagcccaaggctgagagagggacctgagctgagcccagacgatcctgccggcctgctggatctgaggcagggaatgtttgcccagctggtggcccagaatgtgctgctgatcgatggccctctgtcctggtactctgacccaggcctggccggcgtgtctctgaccggaggactgagctataaggaggacacaaaggagctggtggtggccaaggccggcgtgtactacgtgttcttccagctggagctgaggagagtggtggccggcgagggctccggctctgtgagcctggccctgcacctgcagccactgcggagcgccgccggggccgccgccctggccctgaccgtggatctgcctccagcctctagcgaggcacggaacagcgcctttggcttccagggcagactgctgcacctgtccgccggacagaggctgggagtgcacctgcacaccgaggcaagggcccgccacgcatggcagctgacacagggagcaaccgtgctgggcctgttcagagtgacaccagagatccctgccggcctgccaagccctaggtccgagggctctggcgaaggcagaggctccctgctgacttgtggcgacgtggaagaaaatccaggccccaactttctgctgctgacattcatcgtgctgcctctgtgctcccacgccaagttttctatcgtgttcccacagagccagaagggcaactggaagaatgtgccctcctcttaccactattgccctagctcctctgaccagaactggcacaatgatctgctgggcatcacaatgaaggtgaagatgccaaagacccacaaggccatccaggcagatggatggatgtgccacgcagccaagtggatcacaacctgtgactttcggtggtacggccccaagtatatcacccacagcatccactccatccagcctacatccgagcagtgcaaggagtctatcaagcagacaaagcagggaacctggatgagcccaggattcccacctcagaattgtggctacgccacagtgaccgactccgtggcagtggtggtgcaggcaacccctcaccacgtgctggtggatgagtatacaggcgagtggatcgacagccagtttccaaatggcaagtgcgagacagaggagtgtgagaccgtgcacaacagcacagtgtggtactccgattataaggtgacaggcctgtgcgacgccaccctggtggatacagagatcaccttcttttctgaggacggcaagaaggagagcatcggcaagcccaataccggctaccgctccaactacttcgcctatgagaagggcgataaggtgtgcaagatgaactattgtaagcacgccggggtgcggctgccaagcggcgtgtggtttgagttcgtggaccaggacgtgtacgcagcagcaaagctgcctgagtgcccagtgggagcaaccatctccgcccctacacagaccagcgtggacgtgtccctgatcctggatgtggagagaatcctggactacagcctgtgccaggagacatggtctaagatcaggagcaagcagcccgtgtctcctgtggacctgagctatctggcaccaaagaatccaggaaccggaccagcctttacaatcatcaacggcaccctgaagtacttcgagacccggtatatcagaatcgacatcgataatcctatcatcagcaagatggtgggcaagatctccggctctcagacagagagagagctgtggaccgagtggttcccttacgagggcgtggagatcggcccaaacggcatcctgaagacaccaaccggctataagtttcccctgttcatgatcggccacggcatgctggacagcgatctgcacaagacctcccaggccgaggtgtttgagcacccacacctggcagaggcaccaaagcagctgcctgaggaggagacactgttctttggcgataccggcatctctaagaatcccgtggagctgatcgagggctggtttagctcctggaagagcacagtggtgaccttctttttcgccatcggcgtgttcatcctgctgtacgtggtggcaaggatcgtgatcgccgtgcggtacagatatcagggctctaacaataagcggatctataacgacatcgagatgagcaggttccgcaagtga

SEQ ID NO: 5 - DNA, 4590 bp, Mus musculus
atggaccctagatgcacaatgggcctggccatcctgatcttcgtgaccgtgctgctgatcagcgatgccgtgtccgtggagacccaggcctactttaacggcacagcctatctgccatgtcccttcacaaaggcccagaatatctccctgtctgagctggtggtgttttggcaggaccagcagaagctggtgctgtacgagcactatctgggcaccgagaagctggactccgtgaacgccaagtacctgggccggacctcttttgatagaaacaattggacactgaggctgcacaatgtgcagatcaaggatatgggctcttatgactgcttcatccagaagaagccccctaccggcagcatcatcctgcagcagacactgaccgagctgagcgtgatcgccaacttttccgagcccgagatcaagctggcccagaacgtgaccggcaattctggcatcaacctgacatgtaccagcaagcagggccaccctaagccaaagaagatgtacttcctgatcaccaacagcacaaatgagtatggcgacaatatgcagatctcccaggataacgtgaccgagctgtttagcatctccaactctctgagcctgtccttccctgacggcgtgtggcacatgaccgtggtgtgcgtgctggagacagagagcatgaagatcagctccaagcccctgaacttcacccaggagttcccctctcctcagacatactggaaggagatcaccgccagcgtgacagtggccctgctgctggtcatgctgctgatcatcgtgtgccacaagaagccaaatcagcccagccggccttccaacacagcctctaagctggagcgggatagcaatgccgacagagagaccatcaacctgaaggagctggagcctcagatcgcctccgccaagccaaacgcagagggctccggagagggaagaggctctctgctgacatgcggcgacgtggaggagaatccaggacccgccctgccagtgaccgccctgctgctgcctctggccctgctgctgcacgccgccaggccagaggtgcagctggtggagagcggaggaggactggtgcagcctggcaagtctctgaagctgagctgtgaggcctccggctttaccttctccggctacggcatgcactgggtgaggcaggcaccaggaaggggactggagtctgtggcctacatcacatctagctccatcaacatcaagtatgccgacgccgtgaagggccggtttaccgtgagcagagataacgccaagaatctgctgttcctgcagatgaacatcctgaagtccgaggacaccgccatgtactattgcgccagattcgactgggataagaattactggggccagggcaccatggtgacagtgtctagcggaggaggaggctccggaggaggaggctctggcggcggcggcagcgatatccagatgacacagtccccttcctctctgccagcctctctgggcgacagggtgaccatcaactgtcaggcctctcaggatatcagcaactacctgaattggtatcagcagaagcccggcaaggcccctaagctgctgatctactatacaaataagctggccgacggagtgccatcccggttttctggcagcggctccggcagagacagctccttcaccatctctagcctggagtccgaggatatcggctcttactattgccagcagtactataactacccttggaccttcggcccaggcacaaagctggagatcaagcgggaccccagcaccacaacccctgccccaaggccaccaacccccgcccctacaatcgcctcccagccactgtctctgaggcctgaggcctgtcgcccagccgcagggggggcagtgcacaccaggggactggattttgcctgcgacatctacatctgggcacccctggccggaacatgtggcgtgctgctgctgagcctggtcatcaccctgtattgcaagcggggcagaaagaagggcagcggagagggaaggggctccctgctgacctgtggcgacgtggaggagaacccaggccccatggatcagcacaccctggacgtggaggatacagcagacgcaaggcaccccgccggcacatcttgccctagcgatgccgccctgctgagagacaccggactgctggccgatgccgccctgctgtccgacaccgtgcgcccaacaaatgccgccctgccaaccgacgcagcataccctgcagtgaacgtgagggatagggaggcagcatggcctccagccctgaatttttgcagccggcaccctaagctgtatggactggtggccctggtgctgctgctgctgatcgcagcatgcgtgcccatcttcacaaggaccgagcctcgcccagccctgaccatcacaacctcccctaacctgggcacacgcgagaacaatgccgaccaggtgaccccagtgtcccacatcggctgccctaacacaacccagcagggctctccagtgttcgccaagctgctggccaagaatcaggccagcctgtgcaatacaaccctgaactggcactcccaggatggagcaggctcctcttacctgtctcagggcctgcggtatgaggaggacaagaaggagctggtggtggattcccccggcctgtactacgtgttcctggagctgaagctgtctcctacattcaccaatacaggccacaaggtgcagggatgggtgagcctggtgctgcaggccaagcctcaggtggacgattttgacaacctggccctgaccgtggagctgttcccatgctccatggagaataagctggtggatcggtcttggagccagctgctgctgctgaaggcaggacacaggctgagcgtgggactgcgcgcctacctgcacggcgcccaggatgcctacagagactgggagctgtcttatcccaacacaaccagctttggcctgttcctggtgaagccagacaatccatgggaaggctccggcgagggaaggggctctctgctgacctgtggcgatgtggaggagaatcccggccctaactttctgctgctgaccttcatcgtgctgccactgtgcagccacgccaagttttccatcgtgttcccccagtcccagaagggcaactggaagaatgtgcctagctcctaccactattgtccatctagctccgatcagaactggcacaatgacctgctgggcatcaccatgaaggtgaagatgccaaagacacacaaggccatccaggcagacggatggatgtgccacgcagccaagtggatcacaacctgtgattttcgctggtacggccctaagtatatcacacactccatccactctatccagccaaccagcgagcagtgcaaggagtccatcaagcagaccaagcagggaacatggatgagcccaggattcccacctcagaactgtggctacgccaccgtgacagactccgtggcagtggtggtgcaggcaacaccacaccacgtgctggtggacgagtataccggcgagtggatcgatagccagtttcccaacggcaagtgcgagaccgaggagtgtgagacagtgcacaattctaccgtgtggtacagcgactataaggtgaccggcctgtgcgatgccacactggtggacaccgagatcacattctttagcgaggatggcaagaaggagtccatcggcaagcccaataccggctacaggtccaactacttcgcctatgagaagggcgacaaggtgtgcaagatgaattattgtaagcacgccggggtgcggctgcctagcggcgtgtggtttgagttcgtggaccaggacgtgtacgcagcagcaaagctgcctgagtgcccagtgggcgccacaatctctgccccaacccagacaagcgtggacgtgagcctgatcctggacgtggagagaatcctggattacagcctgtgccaggagacctggtctaagatccgcagcaagcagcccgtgtcccctgtggatctgtcttatctggcaccaaagaatccaggaacaggaccagcctttaccatcatcaacggcacactgaagtacttcgagacccggtatatcagaatcgacatcgataaccctatcatcagcaagatggtgggcaagatctccggctctcagaccgagagagagctgtggacagagtggttcccctacgagggcgtggagatcggccctaatggcatcctgaagaccccaacaggctataagtttcccctgttcatgatcggccacggcatgctggactctgatctgcacaagaccagccaggccgaggtgtttgagcacccacacctggcagaggcaccaaagcagctgccagaggaggagaccctgttctttggcgacacaggcatctctaagaaccccgtggagctgatcgagggctggttttctagctggaagagcaccgtggtgacattctttttcgccatcggcgtgttcatcctgctgtacgtggtggcaaggatcgtgatcgccgtgcggtacagatatcagggcagcaacaataagagaatctataacgatatcgagatgtccaggttccgcaagtga

SEQ ID NO: 6 - DNA, 4521 bp, Canis familiaris
atgtacctgcgctgcaccatggagctgaacaatatcctgttcgtgatgacactgctgctgtacggcgccgcctctatgaagagccaggcctacttcaacaagaccggcgagctgccctgtcacttcacaaactcccagaatatctctctggacgagctggtggtgttttggcaggaccaggataagctggtgctgtacgagctgtatcgcggcaaggagaaccctcagaatgtgcacagaaagtacaagggcaggacctcctttgacaaggataactggacactgcggctgcacaatatccagatcaaggataagggcctgtatcagtgcttcgtgcaccacaagggcccaaagggcctggtgcccatgcaccagatgaactccgacctgtctgtgctggccaacttcagccagcccgagatcatggtgacctccaaccggacagagaacagcggcatcatcaacctgacctgtagctccatccagggctaccccgagcctaaggagatgtatttcctggtgaagacagagaacagcagcaccaagtacgatacagtgatgaagaagagccagaacaatgtgaccgagctgtataacgtgtctatcagcctgtccttttctgtgcctgaggccagcaacgtgagcatcttctgcgtgctgcagctggagtctatgaagctgcccagcctgccttacaacatcgacgcccacaccaagccaacacccgacggcgatcacatcctgtggatcgccgccctgctggtcatgctggtcatcctgtgcggcatggtgttctttctgaccctgagaaagcggaagaagaagcagcctggcccatcccacgagtgtgagacaaataaggtggagcgcaaggagtctgagcagaccaaggagcgcgtgcggtaccacgagacagagcggtccgatgaggcccagtgcgtgaacatcagcaagaccgcctccggcgacaattctaccacacagttcggcagcggagagggaagaggctccctgctgacctgtggcgatgtggaggagaacccaggacctgccctgccagtgacagccctgctgctgcctctggccctgctgctgcacgccgccaggccagaggtgcagctggtggagagcggaggaggactggtgcagcccaagggcagcctgaagctgtcctgcgccacctctggctttacattcaacatctacgccatgaattgggtgagacaggcaccaggcaagggactggagtgggtggccagaatcaggagcaagtccaagaattatgccacctactatgccggcagcgtgaaggaccgctttacaatctcccgggacgattctcagagcatgctgtacctgcagatgaacaatctgaagaccgaggatacagccatgtactattgcgtgcggagaggctatttcgacgtgtggggagcaggaaccacagtgaccgtgtcctctagcggaggaggaggctccggaggaggaggctctggcggcggcggcagcatcatcgtgatgacccagtccccaaagtccatgtctatgagcgtgggagagagggtgacactgagctgtcgggcctccgagaacgtggattcttttgtgagctggtaccagcagaagcctgaccagagcccacagctgctgatcttcggcgcctccaatagatctaccggcgtgcccgataggtttaccggctccggctctgccacagacttcacactgaccatcacaagcgtgcagtccgaggatctggcagcataccactgcggacagacctactcctatccttttaccttcggctctggcacaaacctggagatcaagcggagcgaccccaccacaaccccagccccaaggccacctacccctgccccaacaatcgcctctcagcctctgagcctgagaccagaggcctgtaggccagccgcagggggggcagtgcacacccgcggcctggacttcgcctgcgatatctacatctgggcacccctggccggaacctgtggcgtgctgctgctgagcctggtcatcaccctgtattgcaaccaccggaataggcgccgggtgtgcaagtgtccaagacctgtggtgggctctggagagggaaggggcagcctgctgacttgcggcgatgtggaagaaaacccaggaccaatgaggccacggagcgacgccgcccctgatccagaggcaccaaggccacccgcccctccaggcagagcctgcagcccactgccttgggcactgtccgccgcaatgctgctgctggtgggcacctgcgccgcctgtgccctgcgcgcctgggtggtgccaggaccaaggccccctgccctgcccgccctgcctgccccactgcccgatgccggcgccaggctgccagacagcccacaggccgtgttcgcacagctggtggccagagatgtgcagctgaaggagggaccactgaggtggtacagcgaccccggcctggccggcgtgtttctgggacccggcctgtcctatgaccagcacacaagagagctgatggtggtggagcctggcctgtactacgtgttcctgcacctgaagctgcagagagtgatgtcctctaccggcagcggctccgtgtctgccgccctgcacctgcagccactgggaaccgaggcagccgccctggacctgacactggatctgccaccccctagctccgaggccagagattctgccgcaggctttaggggcagcctgctgcacctggacgcaggacagagactgagggtgcacctgagggcagaggcaggagcacaccctgcatggcagctggcccagggagcaacaatcctgggactgttccgggtggcaaccaaggtgccaacaggactgccttctagctggccaatggataccggaccaggctccccaccactggacggagagggctccggcgagggcagaggctctctgctgacctgcggcgacgtggaggagaaccctggaccaaattttctgctgctgacattcatcgtgctgcctctgtgctcccacgccaagttttctatcgtgttcccacagagccagaagggcaactggaagaatgtgccttcctcttaccactattgcccaagctcctctgaccagaactggcacaatgatctgctgggcatcaccatgaaggtgaagatgcctaagacacacaaggccatccaggcagatggatggatgtgccacgcagccaagtggatcacaacctgtgactttaggtggtacggccccaagtatatcacccactctatccacagcatccagcctacaagcgagcagtgcaaggagtccatcaagcagaccaagcagggcacatggatgtctccaggcttccctccacagaactgtggctacgccaccgtgacagacagcgtggcagtggtggtgcaggcaacccctcaccacgtgctggtggatgagtatacaggcgagtggatcgacagccagtttcccaacggcaagtgcgagaccgaggagtgtgagacagtgcacaatagcaccgtgtggtactccgattataaggtgaccggcctgtgcgacgccacactggtggataccgagatcacattcttttccgaggacggcaagaaggagtctatcggcaagcccaacaccggctacagatccaattacttcgcctatgagaagggcgataaggtgtgcaagatgaattattgtaagcacgccggggtgcggctgccaagcggcgtgtggtttgagttcgtggaccaggacgtgtacgcagcagcaaagctgcctgagtgcccagtgggagcaaccatctccgcccccacccagacatccgtggacgtgtctctgatcctggatgtggagcgcatcctggactacagcctgtgccaggagacctggagcaagatccggtccaagcagcccgtgtctcctgtggacctgagctatctggcaccaaagaacccaggaacaggacctgcctttaccatcatcaatggcacactgaagtacttcgagacccgctatatccggatcgacatcgataacccaatcatcagcaagatggtgggcaagatcagcggctcccagaccgagagagagctgtggacagagtggttcccctacgagggcgtggagatcggccctaatggcatcctgaagacccctacaggctataagtttccactgttcatgatcggccacggcatgctggactctgatctgcacaagaccagccaggccgaggtgtttgagcacccacacctggcagaggcaccaaagcagctgcctgaggaggagaccctgttctttggcgatacaggcatctccaagaaccctgtggagctgatcgagggctggtttagctcctggaagtctaccgtggtgacattctttttcgccatcggcgtgttcatcctgctgtacgtggtggccagaatcgtgatcgccgtgcgctaccggtatcagggctctaacaataagaggatctataatgacatcgagatgagcagattcaggaagtga

SEQ ID NO: 7 - protein, 1496 aa, Homo sapiens
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCFGSGEGRGSLLTCGDVEENPGPALPVTALLLPLALLLHAARPQVQLLESGAELARPGASVKMSCKASGYTFTRYTMHWVKQRPGQGLEWIGYINPSRGYTNYNQKFKDKATLTTDKSSSTAYMQLSSLTSEDSAVYYCAGYYDDHYCLDYWGQGTLVTVSSGGGGSGGGGSGGGGSDIVMTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIKRDPSTTTPAPRPPTPAPTIASQPLSLRPEACRPAAGGAVHTRGLDFACDIYIWAPLAGTCGVLLLSLVITLYCKRGRKKGSGEGRGSLLTCGDVEENPGPMEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSGEGRGSLLTCGDVEENPGPNFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 8 - protein, 1529 aa, Mus musculus
MDPRCTMGLAILIFVTVLLISDAVSVETQAYFNGTAYLPCPFTKAQNISLSELVVFWQDQQKLVLYEHYLGTEKLDSVNAKYLGRTSFDRNNWTLRLHNVQIKDMGSYDCFIQKKPPTGSIILQQTLTELSVIANFSEPEIKLAQNVTGNSGINLTCTSKQGHPKPKKMYFLITNSTNEYGDNMQISQDNVTELFSISNSLSLSFPDGVWHMTVVCVLETESMKISSKPLNFTQEFPSPQTYWKEITASVTVALLLVMLLIIVCHKKPNQPSRPSNTASKLERDSNADRETINLKELEPQIASAKPNAEGSGEGRGSLLTCGDVEENPGPALPVTALLLPLALLLHAARPEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKRDPSTTTPAPRPPTPAPTIASQPLSLRPEACRPAAGGAVHTRGLDFACDIYIWAPLAGTCGVLLLSLVITLYCKRGRKKGSGEGRGSLLTCGDVEENPGPMDQHTLDVEDTADARHPAGTSCPSDAALLRDTGLLADAALLSDTVRPTNAALPTDAAYPAVNVRDREAAWPPALNFCSRHPKLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWEGSGEGRGSLLTCGDVEENPGPNFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 9 - protein, 1506 aa, Canis familiaris
MYLRCTMELNNILFVMTLLLYGAASMKSQAYFNKTGELPCHFTNSQNISLDELVVFWQDQDKLVLYELYRGKENPQNVHRKYKGRTSFDKDNWTLRLHNIQIKDKGLYQCFVHHKGPKGLVPMHQMNSDLSVLANFSQPEIMVTSNRTENSGIINLTCSSIQGYPEPKEMYFLVKTENSSTKYDTVMKKSQNNVTELYNVSISLSFSVPEASNVSIFCVLQLESMKLPSLPYNIDAHTKPTPDGDHILWIAALLVMLVILCGMVFFLTLRKRKKKQPGPSHECETNKVERKESEQTKERVRYHETERSDEAQCVNISKTASGDNSTTQFGSGEGRGSLLTCGDVEENPGPALPVTALLLPLALLLHAARPEVQLVESGGGLVQPKGSLKLSCATSGFTFNIYAMNWVRQAPGKGLEWVARIRSKSKNYATYYAGSVKDRFTISRDDSQSMLYLQMNNLKTEDTAMYYCVRRGYFDVWGAGTTVTVSSSGGGGSGGGGSGGGGSIIVMTQSPKSMSMSVGERVTLSCRASENVDSFVSWYQQKPDQSPQLLIFGASNRSTGVPDRFTGSGSATDFTLTITSVQSEDLAAYHCGQTYSYPFTFGSGTNLEIKRSDPTTTPAPRPPTPAPTIASQPLSLRPEACRPAAGGAVHTRGLDFACDIYIWAPLAGTCGVLLLSLVITLYCNHRNRRRVCKCPRPVVGSGEGRGSLLTCGDVEENPGPMRPRSDAAPDPEAPRPPAPPGRACSPLPWALSAAMLLLVGTCAACALRAWVVPGPRPPALPALPAPLPDAGARLPDSPQAVFAQLVARDVQLKEGPLRWYSDPGLAGVFLGPGLSYDQHTRELMVVEPGLYYVFLHLKLQRVMSSTGSGSVSAALHLQPLGTEAAALDLTLDLPPPSSEARDSAAGFRGSLLHLDAGQRLRVHLRAEAGAHPAWQLAQGATILGLFRVATKVPTGLPSSWPMDTGPGSPPLDGEGSGEGRGSLLTCGDVEENPGPNFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 10 - protein, 329 aa, Unknown
  CD86
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 11 - protein, 339 aa, Unknown
  anti-CD3scFv
ALPVTALLLPLALLLHAARPQVQLLESGAELARPGASVKMSCKASGYTFTRYTMHWVKQRPGQGLEWIGYINPSRGYTNYNQKFKDKATLTTDKSSSTAYMQLSSLTSEDSAVYYCAGYYDDHYCLDYWGQGTLVTVSSGGGGSGGGGSGGGGSDIVMTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIKRDPSTTTPAPRPPTPAPTIASQPLSLRPEACRPAAGGAVHTRGLDFACDIYIWAPLAGTCGVLLLSLVITLYCKRGRKK

SEQ ID NO: 12 - protein, 254 aa, Unknown
  CD137L
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 13 - protein, 511 aa, Unknown
  COCVG
NFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 14 - protein, 19 aa, Porcine teschovirus-1
ATNFSLLKQAGDVEENPGP

SEQ ID NO: 15 - protein, 18 aa, Thoseaasigna virus
EGRGSLLTCGDVEENPGP

SEQ ID NO: 16 - protein, 20 aa, Equine rhinitis A virus
QCTNYALLKLAGDCESNPGP

SEQ ID NO: 17 - protein, 22 aa, Foot-and-mouth disease virus
VKQTLNFDLLKLAGDVESNPGP

SEQ ID NO: 18 - DNA, 57 bp, Porcine teschovirus-1
gctactaacttcagcctgctgaagcaggctggagacgtggaggagaaccctggacct

SEQ ID NO: 19 - DNA, 54 bp, Thoseaasigna virus
gagggcagaggaagtctgctaacatgcggtgacgtcgaggagaatcctggacct

SEQ ID NO: 20 - DNA, 60 bp, Equine rhinitis A virus
cagtgtactaattatgctctcttgaaattggctggagatgttgagagcaaccctggacct

SEQ ID NO: 21 - DNA, 66 bp, Foot-and-mouth disease virus
gtgaaacagactttgaattttgaccttctcaagttggcgggagacgtggagtccaaccctggacct

(19) United States
(12) Patent Application Publication
(10) Pub. No.: US 2022/0017920 A1
(43) Pub. Date: Jan. 20, 2022

(54) MULTICISTRONIC VECTOR FOR SURFACE ENGINEERING LENTIVIRAL PARTICLES

(71) Applicant: Umoja Biopharma, Inc., Seattle, WA (US)

(72) Inventors: Andrew SCHARENBERG, Seattle, WA (US); Laurie BEITZ, Seattle, WA (US)

(21) Appl. No.: 17/295,599

(22) PCT Filed: Nov. 21, 2019
(86) PCT No.: PCT/US2019/062675

§ 371 Date: May 20, 2021

Related U.S. Application Data
(60) Provisional application No. 62/770,421, filed on Nov. 21, 2018.

(51) Int. Cl. C12N 15/86; C07K 14/005; C07K 16/28; C07K 14/705

(52) U.S. Cl. CPC C12N 15/86; A61K 39/00; C07K 16/2809; C07K 14/70532; C07K 14/70575; C12N 2740/16043; C12N 2740/16045; C12N 2760/20222; C12N 2740/16052; C12N 2810/6081; C12N 2830/20; C12N 2840/20; C07K 2319/33; C07K 2317/622; C07K 14/005

(57) ABSTRACT

The disclosure relates generally to nucleic acid vectors and packaging cell lines for in vivo expansion of T-cells. More particularly, the disclosure relates to intravenous or intratumoral injection of a lentiviral particle adapted for transduction and expansion of tumor-infiltrating lymphocytes in vivo.

### CROSS-REFERENCE TO RELATED APPLICATION

This application claims priority to U.S. Provisional Patent Application No. 62/770,421, filed Nov. 21, 2018, the disclosure of which is incorporated by reference herein in its entirety for all purposes.

### DESCRIPTION OF THE TEXT FILE SUBMITTED ELECTRONICALLY

The Sequence Listing associated with this application is provided in text format in lieu of a paper copy, and is hereby incorporated by reference into the specification. The name of the text file containing the Sequence Listing is UMOJ-002_01WO_SeqList_ST25.txt. The text file is 99 KB, was created on Nov. 21, 2018, and is being submitted electronically via EFS-Web.

### FIELD OF THE INVENTION

The disclosure relates generally to viral vectors, packaging cell lines, and related methods of use and in particular for expansion of immune cell populations in vivo for treatment of a disease condition.

### BACKGROUND OF THE INVENTION

Cancer immunotherapy is a treatment modality based on therapeutic induction of immune responses to tumors. Adoptive T cell therapy (ACT) is one form of cancer immunotherapy. Lymphocytes, particularly tumor-infiltrating lymphocytes (TILs), are isolated from the body, cultured ex vivo, expanded, and then re-infused. The expansion step may include antigen-specific expansion or genetic engineering of the TILs. ACT is reviewed in Rosenberg et al. Adoptive cell transfer as personalized immunotherapy for human cancer. Science. 348:62-8 (2015).

Certain procedures required by ACT, such as ex vivo expansion of immune cells, are costly, time-consuming, and risky. Accordingly, there is a need for alternative means of expanding populations of TILs.

### SUMMARY OF THE INVENTION

The present disclosure is based, in part, on the discovery that surface-engineered lentiviral particles can be generated using multicistronic vectors designed to express a plurality of polypeptides, namely a fusion glycoprotein or functional variant thereof and one or more non-viral proteins capable of viral surface display. In the multicistronic vectors of the disclosure, the plurality of polypeptides are joined by linkers comprising peptides capable of inducing ribosome skipping or self-cleavage. 2A peptides may be used (e.g. T2A, P2A, E2A, and F2A), or other skipping/self-cleavage peptides.

In one aspect, the disclosure provides a multicistronic vector for surface-engineering lentiviral particles, comprising a polynucleotide operatively linked to a promoter, wherein the polynucleotide encodes a plurality of polypeptides joined by linkers comprising peptides capable of inducing ribosome skipping or self-cleavage, and wherein the plurality of polypeptides comprise a fusion glycoprotein or functional variant thereof and one or more non-viral proteins capable of viral surface display.

In some embodiments, the linkers comprise 2A peptides each independently selected from the group consisting of P2A (SEQ ID NO: 14), T2A (SEQ ID NO: 15), E2A (SEQ ID NO: 16), and F2A (SEQ ID NO: 17).

In some embodiments, one or more of the linkers further comprises a sequence encoding the residues Gly-Ser-Gly.

In some embodiments, the the plurality of polypeptides comprises 2, 3, 4, 5, 6, 7, 8, 9, 10, 11, 12, 13, 14, 15, 16, 17, 18, 19 or 20 proteins capable of viral surface display.

In some embodiments, the fusion glycoprotein or functional variant thereof is a viral fusion glycoprotein or a functional variant thereof.

In some embodiments, the viral fusion glycoprotein or functional variant thereof is Cocal virus G (COCVG) protein or a functional variant thereof.

In some embodiments, the non-viral proteins capable of viral surface display comprise one or more non-viral proteins selected from a transmembrane-domain fusion of a single chain variable fragment (scFv) specific for human CD3 (anti-CD3 scFv), human CD86, and human CD137L, or functional variants thereof.

In some embodiments, the viral fusion glycoprotein is Cocal virus G (COCVG) protein or a functional variant thereof and the plurality of polypeptides comprises the anti-CD3 scFv, the human CD86, and human CD137L, or functional variants thereof.

In some embodiments, the plurality of polypeptides consists of the Cocal virus G (COCVG) protein, the anti-CD3 scFv, the human CD86, and the human CD137L, or functional variants thereof.

In some embodiments, the polynucleotide encodes one of COCVG-2A-anti-CD3scFv-2A-CD86-2A-CD137L; anti-CD3scFv-2A-COCVG-2A-CD86-2A-CD137L; anti-CD3 scFv-2A-CD86-2A-COCVG-2A-CD137L; anti-CD3scFv-2A-CD86-2A-CD137L-2A-COCVG; COCVG-2A-CD86-2A-anti-CD3 scFv-2A-CD137L; CD137L-2A-anti-CD3 scFv-2A-COCVG-2A-CD86; anti-CD3scFv-2A-CD137L-2A-COCVG-2A-CD86; CD86-2A-anti-CD3 scFv-2A-COCVG-2A-CD137L; CD86-2A-COCVG-2A-anti-CD3scFv-2A-CD137L; CD86-2A-COCVG-2A-CD137L-2A-anti-CD3scFv; CD86-2A-anti-CD3 scFv-2A-CD137L-2A-COCVG; CD86-2A-CD137L-2A-anti-CD3 scFv-2A-COCVG; and CD137L-2A-anti-CD3 scFv-2A-CD86-2A-COCVG;

In some embodiments, the polynucleotide encodes CD86-2A-anti-CD3scFv-2A-CD137L-2A-COCVG.

In some embodiments, the mutlicistronic vector is a lentiviral envelope plasmid capable of generating a lentiviral particle pseudotyped for COCV when co-transfected with a transfer plasmid and a packaging plasmid into a packaging cell line.

In another aspect, the disclosure provides a cell comprising a multicistronic vector of the disclosure.

In some embodiments, the cell comprises no other polynucleotides encoding any of the plurality of polypeptides encoded by the multicistronic vector other than the multicistronic vector itself.

In another aspect, the disclosure provides a surface-engineered lentiviral particle produced by a cell of the disclosure. In some embodiments, the lentiviral particle is pseudotyped by the fusion glycoprotein or functional variant thereof. In some embodiments, the lentiviral particle displays on its surface each of the plurality of polypeptides.

In another aspect, the disclosure provides a pharmaceutical composition comprising a surface-engineered lenviral particle of the disclosure.

In another aspect, the disclosure provides a method of generating surface-engineered lentiviral particles, comprising providing a cell in a culture medium; and transfecting the cell with a multicistronic vector of the disclosure, a transfer plasmid, and a packaging plasmid, simultaneously or sequentially; whereby the cell expresses a surface-engineered lentiviral particle.

In some embodiments the titer of surface-engineered lentiviral particle in the culture medium after transfecting the cell is at least about as high as the titer of a pseudotyped lentiviral particle produced by the same method using an pMD2.G plasmid in place of the multicistronic vector.

In some embodiments, the titer of surface-engineered lentiviral particle after step b) is at least about 1×10⁶, 1×10⁷, 2×10⁷, 4×10⁷, 6×10⁷, 8×10⁷, or 1×10⁸ IU/ml.

In some embodiments, the method comprises harvesting the lentiviral particle from the culture medium.

In another aspect, the disclosure provides a method for treating a subject suffering from cancer, comprising administering a surface-engineered lentiviral particle of disclosure to the subject, whereby the cancer is treated in the subject.

In another aspect, the disclosure provides a method for expanding T-cells capable of recognizing and killing tumor cells in a subject in need thereof, comprising administering a lentiviral particle of the disclosure to the subject, whereby T-cells capable of recognizing and killing tumor cells in the subject are transduced by the lentiviral particle and expanded.

In some embodiments, the lentiviral particle is administered by intravenous injection.

In some embodiments, the lentiviral particle is administered by intratumoral injection.

Additional aspects and embodiments of the disclosure will be apparent from the detailed description that follows.

### BRIEF DESCRIPTION OF THE DRAWINGS

FIGS. 1A-1B depict lentiviral particles. FIG. 1A depicts an embodiment of the surface-engineered lentiviral particles of the disclosure, which has surface-expressed anti-CD3 and T-cell co-stimulatory molecules. A conventional lentiviral particle lacking these elements is depicted in FIG. 1B.

FIG. 2 depicts an embodiment of the methods of the disclosure in which a four-vector lentivirus system (often referred to as third-generation lentiviral system) is modified by inclusion of genes encoding co-stimulatory molecules on the same plasmid as the Env gene, and the co-stimulatory molecules and the Env gene are linked by 2A peptides.

FIGS. 3A-3B show two experimental transfer plasmids. FIG. 3A shows mCherry under the control of an MND promoter. FIG. 3B shows the transfer plasmid VT103, which contains a model payload comprising a 2A-linked open-reading frame encoding two proteins that form a dimeric cytokine receptor, a third protein that confers partial resistance to the immunosuppressive agent rapamycin, and a fourth protein that acts as a marker (e.g. a fluorescent protein or a protein detectable by surface staining of cells with an antibody).

FIGS. 4A-4B show titers of lentiviral particles pseudotyped for either Vesicular stomatitis (VSV) or Cocal and surface-engineered with co-stimulatory molecules. The promoter used to express the fusion glycoprotein of VSV or Cocal is indicated by the abbreviations CMV (cytomegalovirus promoter) or MND (a synthetic promoter that contains the U3 region of a modified MoMuLV LTR with myeloproliferative sarcoma virus enhancer). FIG. 4A shows results for monocistronic vectors that express only the VSV or Cocal fusion glycoprotein. FIG. 4B shows results for co-expression from a multicistronic vector of the VSV or Cocal fusion glycoprotein along with T-cell co-activation molecules—CD86, anti-CD3, and CD137L (“stim”).

FIG. 5 depicts results for a monocistronic vector that express the Cocal fusion glycoprotein from an MND or CMV promoter. The transfer plasmids encoded either the mCherry fluorescent maker or a model payload (“VT103”).

FIGS. 6A-6C show design and testing of an embodiment of the multicistronic vectors of the disclosure. FIG. 6A shows an embodiments of the multicistronic vectors of the disclosure where a polynucleotide encoding CD86-2A-anti-CD3scFv-2A-CD137L-2A-COCVG is placed under the control of an MND promoter. FIG. 6B shows an embodiments of the multicistronic vectors of the disclosure where a polynucleotide encoding CD86-2A-anti-CD3scFv-2A-CD137L-2A-COCVG is placed under the control of an CMV promoter.

FIG. 6C shows results for expression of the Cocal fusion glycoprotein from control monocistronic plasmids or with three co-stimulatory molecules—CD86, anti-CD3, and CD137L (“h3stim”)—from multicistronic vectors as shown in FIGS. 6A-6B. The transfer plasmid used was VT103.

FIGS. 7A-7D show human CD3+ cells three days after transduction with lentiviral particles made using mCherry or VT103 transfer plasmids and envelope plasmids encoding either COVG alone or CD86-2A-anti-CD3scFv-2A-CD137L-2A-COCVG at a multiplicity of infection (MOI) of 5. FIG. 7A shows results for mCherry transfer plasmid and COVG without dynabeads. FIG. 7B shows results for mCherry transfer plasmid and COVG with anti-CD3-anti-CD28 dynabeads. FIG. 7C shows results for mCherry transfer plasmid and CD86-2A-anti-CD3scFv-2A-CD137L-2A-COCVG without dynabeads. FIG. 7D shows results for VT103 transfer plasmid and CD86-2A-anti-CD3scFv-2A-CD137L-2A-COCVG without dynabeads.

### DETAILED DESCRIPTION

The present inventors have realized that, as an alternative to ACT, in vivo transduction of TILs, or other immune cells, could potentiate expansion of cells in vivo rather than ex vivo. Furthermore, the present inventors have realized in vivo transduction of TILs, or other immune cells, allows for treatment of cancer or other disease conditions without the drawbacks of ex vivo expansion of immune cells.

Thus, the present disclosure provides means of expanding populations of TILs or other immune cells in vivo. In particular, the present disclosure provides therapeutic agents capable of selectively expanding desirable populations of TILs or other immune cells in vivo. The present disclosure provides viral vectors and related methods of use for expansion of TILs or other immune cells in vivo for treatment of a disease condition.

The present disclosure is based, in part, on the discovery that multicistronic vectors can be used to generate surface-engineered lentiviral particles. Multicistronic vectors designed to express a plurality of polypeptides, namely a fusion glycoprotein or functional variant thereof and one or more non-viral proteins capable of viral surface display are provided. In the multicistronic vectors of the disclosure, the polypeptides are joined by linkers comprising peptides capable of inducing ribosome skipping or self-cleavage. 2A peptides may be used (e.g. T2A, P2A, E2A, and F2A), or alternative skipping/self-cleavage peptides.

Among other uses for the disclosed multicistronic vectors, lentiviral vector systems may be adapted for transduction and expansion of tumor-infiltrating lymphocytes in vivo by use of the disclosed multicistronic vectors. In some embodiments, the disclosed multicistronic vectors generate surface-engineered lentiviral particles that provide a co-stimulatory effect on target cells (e.g., T cells and/or NK-cells). Thus, cells that ordinarily cannot be efficiently transduced in vivo may be transduced by the surface-engineered lentiviral particles of the disclosure.

In some embodiments, the vectors and particles of the disclosure are configured for use in vivo. The multicistronic vectors and lentiviral particles disclosed can also be used for ACT by in vitro transduction of autologous or allogeneic T cells. In some embodiments, the vectors and particles of the disclosure are configured for use in vitro.

### 1.1 Nucleic Acid Vectors

As used herein, the term “nucleic acid vector” is intended to mean any nucleic acid that functions to carry, harbor, or express a nucleic acid of interest. Nucleic acid vectors can have specialized functions such as expression, packaging, pseudotyping, or transduction. Nucleic acid vectors also can have manipulatory functions if adapted for use as a cloning or shuttle vector. The structure of the vector can include any desired form that is feasible to make and desirable for a particular use. Such forms include, for example, circular forms such as plasmids and phagemids, as well as linear or branched forms. A nucleic acid vector can be composed of, for example, DNA or RNA, as well as contain partially or fully, nucleotide derivatives, analogs and mimetics. Such nucleic acid vectors can be obtained from natural sources, produced recombinantly or chemically synthesized.

Non-limiting examples of vector systems of the present disclosure include a retrovirus, a lentivius, a foamy virus, and a Sleeping Beauty transposon.

### 1.2 Lentiviral Vector Systems

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV-1 and HIV-2) and the Simian Immunodeficiency Virus (SIV). Lentiviral vector systems have been generated by multiply attenuating the HIV virulence genes, for example, the genes env, vif, vpr, vpu and nef are deleted, making the vector biologically safe.

Lentiviral vector systems offer great advantages for gene therapy. Unless engineered to be non-integrating, lentiviral vectors integrate stably into chromosomes of target cells, permitting long-term expression of delivered transgenes. Further, they do not transfer viral genes therefore avoiding the problem of generating transduced cells that can be destroyed by cytotoxic T-cells. Furthermore, they have a relatively large cloning capacity, sufficient for most envisioned clinical applications. In addition, lentiviruses, in contrast to other retroviruses, are capable of transducing non-dividing cells. This is very important in the context of gene-therapy for tissues such as the hematopoietic system, the brain, liver, lungs and muscle. For example, vectors derived from HIV-1 allow efficient in vivo and ex vivo delivery, integration and stable expression of transgenes into cells such as neurons, hepatocytes, and myocytes (Blomer et al., 1997; Kafri et al., 1997; Naldini et al., 1996; Naldini et al., 1998).

Lentiviral vector systems are known in the art, see Naldini et al., (1996) Science 272:263-7; Zufferey et al., (1998) J. Virol. 72:9873-9880; Dull et al., (1998) J. Virol. 72:8463-8471, U.S. Pat. Nos. 6,013,516; and 5,994,136, which are each incorporated herein by reference in its entirety. In general, these vectors are plasmid-based or virus-based, and are configured to carry the essential sequences for selection of cells containing the vector, for incorporating nucleic acid into a lentiviral particle, and for transfer of the nucleic acid into a target cell.

A commonly used lentiviral vector system is the so-called third-generation system. Third-generation lenviral vector systems include four plasmids. The “transfer plasmid” encodes the polynucleotide sequence that is delivered by the lentiviral vector system to the target cell. The transfer plasmid generally has one or more transgene sequences of interest flanked by long terminal repeat (LTR) sequences, which facilitate integration of the transfer plasmid sequences into the host genome. For safety reasons, transfer plasmids are generally designed to make the resulting vector replication incompetent. For example, the transfer plasmid lacks gene elements necessary for generation of infective particles in the host cell. In addition, the transfer plasmid may be designed with a deletion of the 3′ LTR, rendering the virus “self-inactivating” (SIN). See Dull et al. J. Virol. 72:8463-71 (1998); Miyoshi et al. J. Virol. 72:8150-57 (1998).

Third-generation systems also generally include two “packaging plasmids” and an “envelope plasmid.” The “envelope plasmid” generally encodes an Env gene operatively linked to a promoter. In an exemplary third-generation system, the Env gene is VSV-G and the promoter is the CMV promoter. The third-generation system uses two packaging plasmids, one encoding gag and pol and the other encoding rev as a further safety feature—an improvement over the single packaging plasmid of so-called second-generation systems. Although safer, the third-generation system can be more cumbersome to use and result in lower viral titers due to the addition of an additional plasmid. Exemplary packing plasmids include, without limitation, pMD2.G, pRSV-rev, pMDLG-pRRE, and pRRL-GOI.

As used herein, the term “lentiviral vector” is intended to mean a nucleic acid that encodes a lentiviral cis nucleic acid sequence required for genome packaging. A lentiviral vector also can encode other cis nucleic acid sequences beneficial for gene delivery, including for example, cis sequences required for reverse transcription, proviral integration or genome transcription. A lentiviral vector performs transduction functions of a lentiviral vector. As such, the exact makeup of a vector genome will depend on the genetic material desired to be introduced into a target cell. Therefore, a vector genome can encode, for example, additional polypeptides or functions other than that required for packaging, reverse transcription, integration, or transcription. Such functions generally include coding for cis elements required for expression of a nucleic acid of interest. The lentiviral cis sequences or elements can be derived from a lentivirus genome or other virus or vector genome so long as the lentiviral vector genome can be packaged by a packaging cell line into a lentiviral particle and introduced into a target cell.

Lentiviral vector systems rely on the use of a “packaging cell line.” In general, the packaging cell line is a cell line whose cells are capable of producing infectious lentiviral particles when the transfer plasmid, packaging plasmid(s), and envelope plasmid are introduced into the cells. Various methods of introducing the plasmids into the cells may be used, including transfection or electroporation. In some embodiments, a packaging cell line is adapted for high-efficiency packaging of a lentiviral vector system into lentiviral particles.

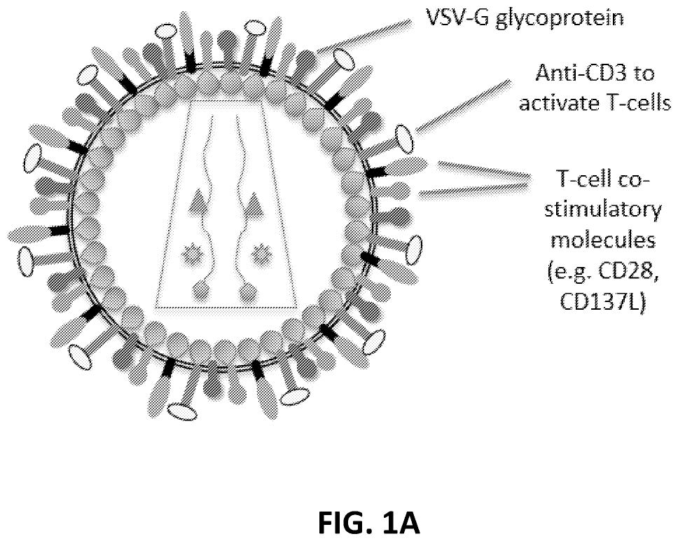

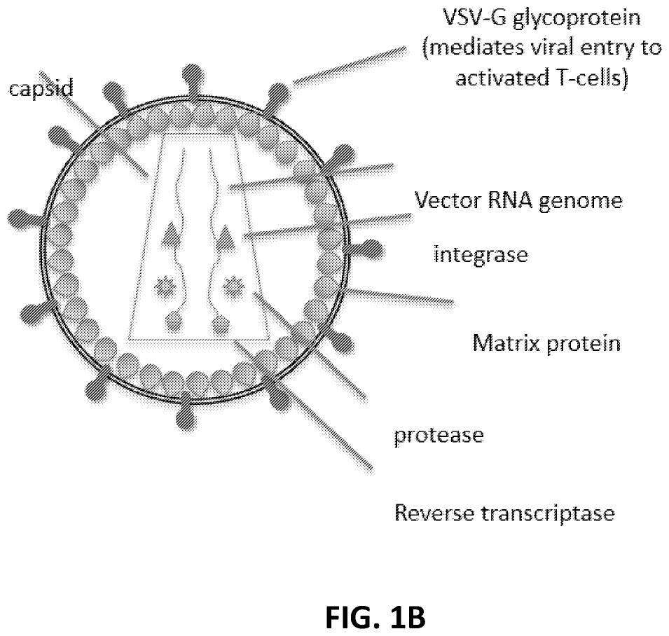

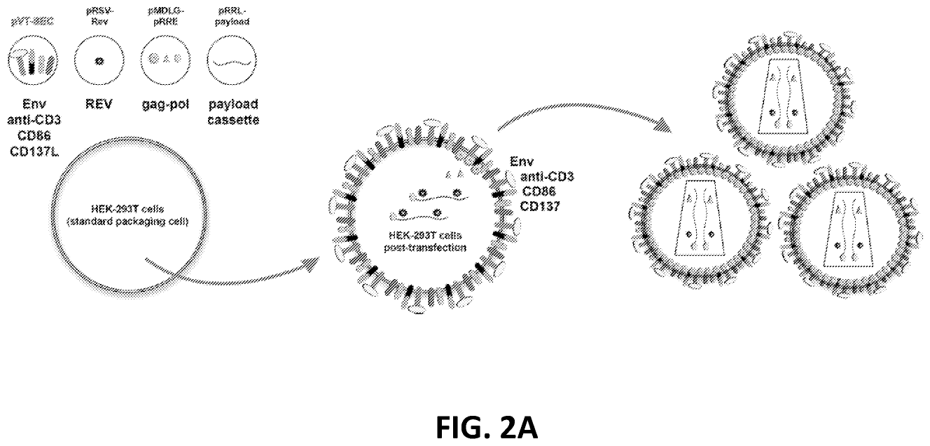

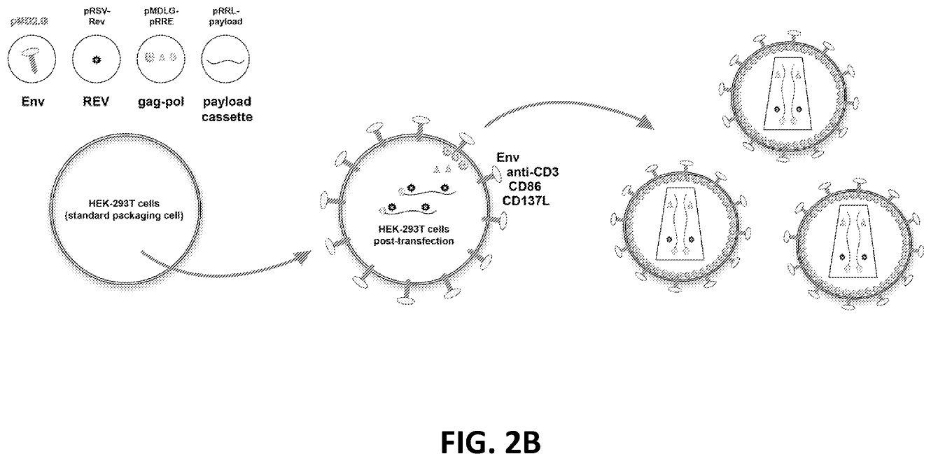

The lentiviral particles produced generally include an RNA genome (derived from the transfer plasmid), a lipid-bilayer envelope in which the Env protein is embedded, and other accessory proteins including integrase, protease, and matrix protein (see FIG. 1B). As used herein, the term “lentiviral particle” is intended to mean a viral particle that includes an envelope, has one or more characteristics of a lentivirus, and is capable of invading a target host cell. Such characteristics include, for example, infecting non-dividing host cells, transducing non-dividing host cells, infecting or transducing host immune cells, containing a lentiviral virion including one or more of the gag structural polypeptides p7, p24, and p17, containing a lentiviral envelope including one or more of the env encoded glycoproteins p41, p120, and p160, containing a genome including one or more lentivirus cis-acting sequences functioning in replication, proviral integration or transcription, containing a genome encoding a lentiviral protease, reverse transcriptase or integrase, or containing a genome encoding regulatory activities such as Tat or Rev. The transfer plasmids may comprise a cPPT sequence, as described in U.S. Pat. No. 8,093,042.

The efficiency of the system is an important concern in vector engineering. The efficiency of a lentiviral vector system may be assessed in various ways known in the art, including measurement of vector copy number (VCN) or vector genomes (vg) such as by quantitative polymerase chain reaction (qPCR), or titer of the virus in infectious units per milliliter (IU/mL). For example, the titer may be assessed using a functional assay performed on the cultured tumor cell line HT1080 as described in Humbert et al. Development of Third-generation Cocal Envelope Producer Cell Lines for Robust Lentiviral Gene Transfer into Hematopoietic Stem Cells and T-cells. Molecular Therapy 24:1237-1246 (2016). When titer is assessed on a cultured cell line that is continually dividing, no stimulation is required and hence the measured titer is not influenced by surface engineering of the lentiviral particle. Other methods for assessing the efficiency of lentiviral vector systems are provided in Gaererts et al. BMC Biotechnol. 6:34 (2006).

It is widely known that lentiviral vector systems have limited efficiency and that attempts to alter the lentiviral vector system often result in decreased efficiency. The present inventors have surprisingly discovered that the envelope plasmid of lentiviral vector systems (e.g. a third-generation system) can be modified to encode a plurality of polypeptides in addition to the fusion glycoprotein or functional variant thereof.

In some embodiments, the multicistronic vectors of the disclosure are capable of generating surface-engineered lentiviral particles at titres of at least about 1×10⁶ IU/mL, at least about 2×10⁶ IU/mL, at least about 3×10⁶ IU/mL, at least about 4×10⁶ IU/mL, at least about 5×10⁶ IU/mL, at least about 6×10⁶ IU/mL, at least about 7×10⁶ IU/mL, at least about 8×10⁶ IU/mL, at least about 9×10⁶ IU/mL, or at least about 1×10⁷ IU/mL. In some embodiments, the multicistronic vectors of the disclosure are capable of generating surface-engineered lentiviral particles at titres of at least about 1×10⁷ IU/mL, at least about 2×10⁷ IU/mL, at least about 3×10⁷ IU/mL, at least about 4×10⁷ IU/mL, at least about 5×10⁷ IU/mL, at least about 6×10⁷ IU/mL, at least about 7×10⁷ IU/mL, at least about 8×10⁷ IU/mL, at least about 9×10⁷ IU/mL, or at least about 1×10⁸ IU/mL. In some embodiments, the multicistronic vector comprises 2, 3, 4, 5, 6, 7, 8, 9, 10, 11, 12, 13, 14, 15, 16, 17, 18, 19 or 20 proteins capable of viral surface display.

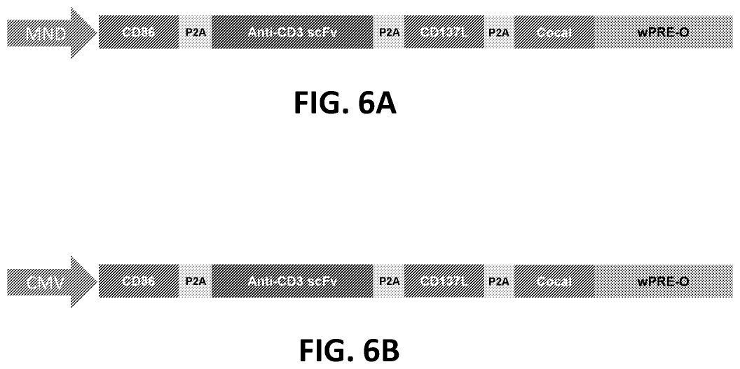

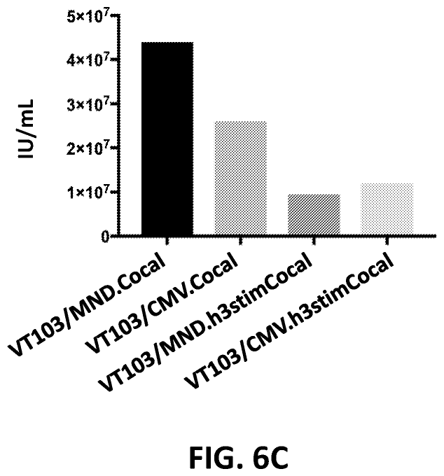

Non-limiting examples of multicistronic vectors of the disclosure include SEQ ID NOs: 1-3, which are illustrated in FIGS. 6-11. In some embodiments, the sequence is codon-optimized.

In some embodiments, the polynucleotide of the multicistronic vector encodes CD86-2A-anti-CD3 scFv-2A-CD137L-2A-COCVG. In some embodiments, the CD86-2A-anti-CD3scFv-2A-CD137L-2A-COCVG comprises polypeptides homologous to the sequences of the respective human, canine, or mouse proteins. In some embodiments, the polynucleotide sequence of the polynucleotide encoding the CD86-2A-anti-CD3scFv-2A-CD137L-2A-COCVG shares at least 75%, 80%, 85%, 90%, 95%, 99%, or 100% identity with one of SEQ ID NOs: 4-6. In some embodiments, the protein sequence of the CD86-2A-anti-CD3scFv-2A-CD137L-2A-COCVG shares at least 75%, 80%, 85%, 90%, 95%, 99%, or 100% identity with one of SEQ ID NOs: 7-9. The following examples use the Thoseaasigna virus (T2A) linker (SEQ ID NO: 15) (bold and underlined) with an N-terminal Gly-Ser-Gly motif (bold).

Non-limiting example of human CD86-2A-anti-
CD3scFv-2A-CD137L-2A-COCVG (polynucleotide
sequence):
(SEQ ID NO: 4)
ATGGACCCCCAGTGCACCATGGGCCTGTCTAATATCCTGTTTGTGATGGC
CTTCCTGCTGAGCGGAGCAGCACCACTGAAGATCCAGGCCTACTTTAACG
AGACAGCCGACCTGCCCTGTCAGTTCGCCAACTCCCAGAATCAGTCTCTG
AGCGAGCTGGTGGTGTTCTGGCAGGATCAGGAGAACCTGGTGCTGAATGA
GGTGTACCTGGGCAAGGAGAAGTTTGACAGCGTGCACTCCAAGTATATGG
GCCGGACCAGCTTCGACTCCGATTCTTGGACCCTGAGGCTGCACAATCTG
CAGATCAAGGATAAGGGCCTGTACCAGTGCATCATCCACCACAAGAAGCC
TACCGGCATGATCAGAATCCACCAGATGAACAGCGAGCTGAGCGTGCTGG
CCAACTTTTCCCAGCCTGAGATCGTGCCAATCTCTAACATCACAGAGAAC
GTGTACATCAACCTGACCTGTAGCTCCATCCACGGCTATCCAGAGCCCAA
GAAGATGAGCGTGCTGCTGAGGACAAAGAACAGCACCATCGAGTACGACG
GCATCATGCAGAAGTCCCAGGATAACGTGACCGAGCTGTATGACGTGAGC
ATCTCCCTGTCTGTGAGCTTTCCAGATGTGACATCCAACATGACCATCTT
CTGCATCCTGGAGACAGACAAGACCCGCCTGCTGTCTAGCCCATTTTCTA
TCGAGCTGGAGGACCCCCAGCCACCTCCAGATCACATCCCTTGGATCACA
GCCGTGCTGCCAACCGTGATCATCTGCGTGATGGTGTTCTGTCTGATCCT
GTGGAAGTGGAAGAAGAAGAAGCGGCCTCGCAATTCCTACAAGTGTGGCA
CAAACACCATGGAGCGGGAGGAGTCTGAGCAGACCAAGAAGAGAGAGAAG
ATCCACATCCCAGAGCGGAGCGATGAGGCCCAGAGAGTGTTTAAGTCCTC
TAAGACAAGCTCCTGCGACAAGTCCGATACCTGTTTCGGCTCTGGAGAGG
GAAGGGGCAGCCTGCTGACCTGCGGCGACGTGGAGGAGAATCCTGGACCA
GCCCTGCCAGTGACAGCCCTGCTGCTGCCTCTGGCCCTGCTGCTGCACGC
CGCCCGCCCTCAGGTGCAGCTGCTGGAGTCCGGAGCAGAGCTGGCCCGGC
CAGGAGCCTCTGTGAAGATGAGCTGTAAGGCCTCCGGCTACACCTTCACC
AGGTATACCATGCACTGGGTGAAGCAGAGGCCAGGACAGGGACTGGAGTG
GATCGGCTACATCAACCCCAGCCGCGGCTACACAAACTATAATCAGAAGT
TCAAGGATAAGGCCACACTGACCACAGACAAGTCTAGCTCCACCGCCTAC
ATGCAGCTGTCTAGCCTGACATCCGAGGATTCTGCCGTGTACTATTGCGC
CGGCTACTATGACGATCACTACTGTCTGGACTATTGGGGCCAGGGCACAC
TGGTGACCGTGTCCTCTGGAGGAGGAGGCTCCGGCGGAGGAGGCTCTGGC
GGCGGCGGCAGCGATATCGTGATGACCCAGTCCCCAGCCATCATGTCCGC
CTCTCCAGGAGAGAAGGTGACAATGACCTGCTCCGCCAGCTCCAGCGTGA
GCTACATGAATTGGTATCAGCAGAAGAGCGGCACCTCCCCCAAGAGATGG
ATCTACGACACATCTAAGCTGGCCAGCGGAGTGCCTGCACACTTCAGGGG
CAGCGGCTCCGGCACATCTTATAGCCTGACCATCAGCGGAATGGAGGCAG
AGGATGCAGCAACCTACTATTGCCAGCAGTGGAGCTCCAACCCCTTCACC
TTCGGCAGCGGCACCAAGCTGGAGATCAAGCGCGACCCCTCTACCACAAC
CCCCGCCCCTAGGCCACCTACACCAGCCCCAACCATCGCCTCCCAGCCAC
TGTCTCTGAGGCCCGAGGCCTGTCGCCCTGCCGCAGGGGGGGCAGTGCAC
ACCAGGGGACTGGACTTTGCCTGCGATATCTACATCTGGGCACCTCTGGC
CGGAACCTGTGGCGTGCTGCTGCTGAGCCTGGTCATCACCCTGTATTGCA
AGCGGGGCAGAAAGAAGGGCAGCGGAGAGGGAAGGGGCTCCCTGCTGACC
TGTGGCGACGTGGAGGAGAACCCTGGCCCAATGGAGTACGCCTCTGACGC
CAGCCTGGACCCCGAGGCCCCATGGCCACCCGCCCCAAGGGCAAGGGCCT
GCCGGGTGCTGCCTTGGGCCCTGGTGGCCGGCCTGTTATTACTGCTGCTG
CTGGCCGCCGCCTGCGCCGTGTTCCTGGCCTGTCCCTGGGCCGTGTCCGG
CGCCAGAGCCTCCCCAGGCTCTGCCGCCAGCCCAAGGCTGAGAGAGGGAC
CTGAGCTGAGCCCAGACGATCCTGCCGGCCTGCTGGATCTGAGGCAGGGA
ATGTTTGCCCAGCTGGTGGCCCAGAATGTGCTGCTGATCGATGGCCCTCT
GTCCTGGTACTCTGACCCAGGCCTGGCCGGCGTGTCTCTGACCGGAGGAC
TGAGCTATAAGGAGGACACAAAGGAGCTGGTGGTGGCCAAGGCCGGCGTG
TACTACGTGTTCTTCCAGCTGGAGCTGAGGAGAGTGGTGGCCGGCGAGGG
CTCCGGCTCTGTGAGCCTGGCCCTGCACCTGCAGCCACTGCGGAGCGCCG
CCGGGGCCGCCGCCCTGGCCCTGACCGTGGATCTGCCTCCAGCCTCTAGC
GAGGCACGGAACAGCGCCTTTGGCTTCCAGGGCAGACTGCTGCACCTGTC
CGCCGGACAGAGGCTGGGAGTGCACCTGCACACCGAGGCAAGGGCCCGCC
ACGCATGGCAGCTGACACAGGGAGCAACCGTGCTGGGCCTGTTCAGAGTG
ACACCAGAGATCCCTGCCGGCCTGCCAAGCCCTAGGTCCGAGGGCTCTGG
CGAAGGCAGAGGCTCCCTGCTGACTTGTGGCGACGTGGAAGAAAATCCAG
GCCCCAACTTTCTGCTGCTGACATTCATCGTGCTGCCTCTGTGCTCCCAC
GCCAAGTTTTCTATCGTGTTCCCACAGAGCCAGAAGGGCAACTGGAAGAA
TGTGCCCTCCTCTTACCACTATTGCCCTAGCTCCTCTGACCAGAACTGGC
ACAATGATCTGCTGGGCATCACAATGAAGGTGAAGATGCCAAAGACCCAC
AAGGCCATCCAGGCAGATGGATGGATGTGCCACGCAGCCAAGTGGATCAC
AACCTGTGACTTTCGGTGGTACGGCCCCAAGTATATCACCCACAGCATCC
ACTCCATCCAGCCTACATCCGAGCAGTGCAAGGAGTCTATCAAGCAGACA
AAGCAGGGAACCTGGATGAGCCCAGGATTCCCACCTCAGAATTGTGGCTA
CGCCACAGTGACCGACTCCGTGGCAGTGGTGGTGCAGGCAACCCCTCACC
ACGTGCTGGTGGATGAGTATACAGGCGAGTGGATCGACAGCCAGTTTCCA
AATGGCAAGTGCGAGACAGAGGAGTGTGAGACCGTGCACAACAGCACAGT
GTGGTACTCCGATTATAAGGTGACAGGCCTGTGCGACGCCACCCTGGTGG
ATACAGAGATCACCTTCTTTTCTGAGGACGGCAAGAAGGAGAGCATCGGC
AAGCCCAATACCGGCTACCGCTCCAACTACTTCGCCTATGAGAAGGGCGA
TAAGGTGTGCAAGATGAACTATTGTAAGCACGCCGGGGTGCGGCTGCCAA
GCGGCGTGTGGTTTGAGTTCGTGGACCAGGACGTGTACGCAGCAGCAAAG
CTGCCTGAGTGCCCAGTGGGAGCAACCATCTCCGCCCCTACACAGACCAG
CGTGGACGTGTCCCTGATCCTGGATGTGGAGAGAATCCTGGACTACAGCC
TGTGCCAGGAGACATGGTCTAAGATCAGGAGCAAGCAGCCCGTGTCTCCT
GTGGACCTGAGCTATCTGGCACCAAAGAATCCAGGAACCGGACCAGCCTT
TACAATCATCAACGGCACCCTGAAGTACTTCGAGACCCGGTATATCAGAA
TCGACATCGATAATCCTATCATCAGCAAGATGGTGGGCAAGATCTCCGGC
TCTCAGACAGAGAGAGAGCTGTGGACCGAGTGGTTCCCTTACGAGGGCGT
GGAGATCGGCCCAAACGGCATCCTGAAGACACCAACCGGCTATAAGTTTC
CCCTGTTCATGATCGGCCACGGCATGCTGGACAGCGATCTGCACAAGACC
TCCCAGGCCGAGGTGTTTGAGCACCCACACCTGGCAGAGGCACCAAAGCA
GCTGCCTGAGGAGGAGACACTGTTCTTTGGCGATACCGGCATCTCTAAGA
ATCCCGTGGAGCTGATCGAGGGCTGGTTTAGCTCCTGGAAGAGCACAGTG
GTGACCTTCTTTTTCGCCATCGGCGTGTTCATCCTGCTGTACGTGGTGGC
AAGGATCGTGATCGCCGTGCGGTACAGATATCAGGGCTCTAACAATAAGC
GGATCTATAACGACATCGAGATGAGCAGGTTCCGCAAGTGA
Non-limiting example of murine CD86-2A-anti-
CD3scFv-2A-CD137L-2A-COCVG (polynucleotide
sequence):
(SEQ ID NO: 5)
ATGGACCCTAGATGCACAATGGGCCTGGCCATCCTGATCTTCGTGACCGT
GCTGCTGATCAGCGATGCCGTGTCCGTGGAGACCCAGGCCTACTTTAACG
GCACAGCCTATCTGCCATGTCCCTTCACAAAGGCCCAGAATATCTCCCTG
TCTGAGCTGGTGGTGTTTTGGCAGGACCAGCAGAAGCTGGTGCTGTACGA
GCACTATCTGGGCACCGAGAAGCTGGACTCCGTGAACGCCAAGTACCTGG
GCCGGACCTCTTTTGATAGAAACAATTGGACACTGAGGCTGCACAATGTG
CAGATCAAGGATATGGGCTCTTATGACTGCTTCATCCAGAAGAAGCCCCC
TACCGGCAGCATCATCCTGCAGCAGACACTGACCGAGCTGAGCGTGATCG
CCAACTTTTCCGAGCCCGAGATCAAGCTGGCCCAGAACGTGACCGGCAAT
TCTGGCATCAACCTGACATGTACCAGCAAGCAGGGCCACCCTAAGCCAAA
GAAGATGTACTTCCTGATCACCAACAGCACAAATGAGTATGGCGACAATA
TGCAGATCTCCCAGGATAACGTGACCGAGCTGTTTAGCATCTCCAACTCT
CTGAGCCTGTCCTTCCCTGACGGCGTGTGGCACATGACCGTGGTGTGCGT
GCTGGAGACAGAGAGCATGAAGATCAGCTCCAAGCCCCTGAACTTCACCC
AGGAGTTCCCCTCTCCTCAGACATACTGGAAGGAGATCACCGCCAGCGTG
ACAGTGGCCCTGCTGCTGGTCATGCTGCTGATCATCGTGTGCCACAAGAA
GCCAAATCAGCCCAGCCGGCCTTCCAACACAGCCTCTAAGCTGGAGCGGG
ATAGCAATGCCGACAGAGAGACCATCAACCTGAAGGAGCTGGAGCCTCAG
ATCGCCTCCGCCAAGCCAAACGCAGAGGGCTCCGGAGAGGGAAGAGGCTC
TCTGCTGACATGCGGCGACGTGGAGGAGAATCCAGGACCCGCCCTGCCAG
TGACCGCCCTGCTGCTGCCTCTGGCCCTGCTGCTGCACGCCGCCAGGCCA
GAGGTGCAGCTGGTGGAGAGCGGAGGAGGACTGGTGCAGCCTGGCAAGTC
TCTGAAGCTGAGCTGTGAGGCCTCCGGCTTTACCTTCTCCGGCTACGGCA
TGCACTGGGTGAGGCAGGCACCAGGAAGGGGACTGGAGTCTGTGGCCTAC
ATCACATCTAGCTCCATCAACATCAAGTATGCCGACGCCGTGAAGGGCCG
GTTTACCGTGAGCAGAGATAACGCCAAGAATCTGCTGTTCCTGCAGATGA
ACATCCTGAAGTCCGAGGACACCGCCATGTACTATTGCGCCAGATTCGAC
TGGGATAAGAATTACTGGGGCCAGGGCACCATGGTGACAGTGTCTAGCGG
AGGAGGAGGCTCCGGAGGAGGAGGCTCTGGCGGCGGCGGCAGCGATATCC
AGATGACACAGTCCCCTTCCTCTCTGCCAGCCTCTCTGGGCGACAGGGTG
ACCATCAACTGTCAGGCCTCTCAGGATATCAGCAACTACCTGAATTGGTA
TCAGCAGAAGCCCGGCAAGGCCCCTAAGCTGCTGATCTACTATACAAATA
AGCTGGCCGACGGAGTGCCATCCCGGTTTTCTGGCAGCGGCTCCGGCAGA
GACAGCTCCTTCACCATCTCTAGCCTGGAGTCCGAGGATATCGGCTCTTA
CTATTGCCAGCAGTACTATAACTACCCTTGGACCTTCGGCCCAGGCACAA
AGCTGGAGATCAAGCGGGACCCCAGCACCACAACCCCTGCCCCAAGGCCA
CCAACCCCCGCCCCTACAATCGCCTCCCAGCCACTGTCTCTGAGGCCTGA
GGCCTGTCGCCCAGCCGCAGGGGGGGCAGTGCACACCAGGGGACTGGATT
TTGCCTGCGACATCTACATCTGGGCACCCCTGGCCGGAACATGTGGCGTG
CTGCTGCTGAGCCTGGTCATCACCCTGTATTGCAAGCGGGGCAGAAAGAA
GGGCAGCGGAGAGGGAAGGGGCTCCCTGCTGACCTGTGGCGACGTGGAGG
AGAACCCAGGCCCCATGGATCAGCACACCCTGGACGTGGAGGATACAGCA
GACGCAAGGCACCCCGCCGGCACATCTTGCCCTAGCGATGCCGCCCTGCT
GAGAGACACCGGACTGCTGGCCGATGCCGCCCTGCTGTCCGACACCGTGC
GCCCAACAAATGCCGCCCTGCCAACCGACGCAGCATACCCTGCAGTGAAC
GTGAGGGATAGGGAGGCAGCATGGCCTCCAGCCCTGAATTTTTGCAGCCG
GCACCCTAAGCTGTATGGACTGGTGGCCCTGGTGCTGCTGCTGCTGATCG
CAGCATGCGTGCCCATCTTCACAAGGACCGAGCCTCGCCCAGCCCTGACC
ATCACAACCTCCCCTAACCTGGGCACACGCGAGAACAATGCCGACCAGGT
GACCCCAGTGTCCCACATCGGCTGCCCTAACACAACCCAGCAGGGCTCTC
CAGTGTTCGCCAAGCTGCTGGCCAAGAATCAGGCCAGCCTGTGCAATACA
ACCCTGAACTGGCACTCCCAGGATGGAGCAGGCTCCTCTTACCTGTCTCA
GGGCCTGCGGTATGAGGAGGACAAGAAGGAGCTGGTGGTGGATTCCCCCG
GCCTGTACTACGTGTTCCTGGAGCTGAAGCTGTCTCCTACATTCACCAAT
ACAGGCCACAAGGTGCAGGGATGGGTGAGCCTGGTGCTGCAGGCCAAGCC
TCAGGTGGACGATTTTGACAACCTGGCCCTGACCGTGGAGCTGTTCCCAT
GCTCCATGGAGAATAAGCTGGTGGATCGGTCTTGGAGCCAGCTGCTGCTG
CTGAAGGCAGGACACAGGCTGAGCGTGGGACTGCGCGCCTACCTGCACGG
CGCCCAGGATGCCTACAGAGACTGGGAGCTGTCTTATCCCAACACAACCA
GCTTTGGCCTGTTCCTGGTGAAGCCAGACAATCCATGGGAAGGCTCCGGC
GAGGGAAGGGGCTCTCTGCTGACCTGTGGCGATGTGGAGGAGAATCCCGG
CCCTAACTTTCTGCTGCTGACCTTCATCGTGCTGCCACTGTGCAGCCACG
CCAAGTTTTCCATCGTGTTCCCCCAGTCCCAGAAGGGCAACTGGAAGAAT
GTGCCTAGCTCCTACCACTATTGTCCATCTAGCTCCGATCAGAACTGGCA
CAATGACCTGCTGGGCATCACCATGAAGGTGAAGATGCCAAAGACACACA
AGGCCATCCAGGCAGACGGATGGATGTGCCACGCAGCCAAGTGGATCACA
ACCTGTGATTTTCGCTGGTACGGCCCTAAGTATATCACACACTCCATCCA
CTCTATCCAGCCAACCAGCGAGCAGTGCAAGGAGTCCATCAAGCAGACCA
AGCAGGGAACATGGATGAGCCCAGGATTCCCACCTCAGAACTGTGGCTAC
GCCACCGTGACAGACTCCGTGGCAGTGGTGGTGCAGGCAACACCACACCA
CGTGCTGGTGGACGAGTATACCGGCGAGTGGATCGATAGCCAGTTTCCCA
ACGGCAAGTGCGAGACCGAGGAGTGTGAGACAGTGCACAATTCTACCGTG
TGGTACAGCGACTATAAGGTGACCGGCCTGTGCGATGCCACACTGGTGGA
CACCGAGATCACATTCTTTAGCGAGGATGGCAAGAAGGAGTCCATCGGCA
AGCCCAATACCGGCTACAGGTCCAACTACTTCGCCTATGAGAAGGGCGAC
AAGGTGTGCAAGATGAATTATTGTAAGCACGCCGGGGTGCGGCTGCCTAG
CGGCGTGTGGTTTGAGTTCGTGGACCAGGACGTGTACGCAGCAGCAAAGC
TGCCTGAGTGCCCAGTGGGCGCCACAATCTCTGCCCCAACCCAGACAAGC
GTGGACGTGAGCCTGATCCTGGACGTGGAGAGAATCCTGGATTACAGCCT
GTGCCAGGAGACCTGGTCTAAGATCCGCAGCAAGCAGCCCGTGTCCCCTG
TGGATCTGTCTTATCTGGCACCAAAGAATCCAGGAACAGGACCAGCCTTT
ACCATCATCAACGGCACACTGAAGTACTTCGAGACCCGGTATATCAGAAT
CGACATCGATAACCCTATCATCAGCAAGATGGTGGGCAAGATCTCCGGCT
CTCAGACCGAGAGAGAGCTGTGGACAGAGTGGTTCCCCTACGAGGGCGTG
GAGATCGGCCCTAATGGCATCCTGAAGACCCCAACAGGCTATAAGTTTCC
CCTGTTCATGATCGGCCACGGCATGCTGGACTCTGATCTGCACAAGACCA
GCCAGGCCGAGGTGTTT GAGCACCCACACCTGGCAGAGGCACCAAAGCAG
CTGCCAGAGGAGGAGACCCTGTTCTTTGGCGACACAGGCATCTCTAAGAA
CCCCGTGGAGCTGATCGAGGGCTGGTTTTCTAGCTGGAAGAGCACCGTGG
TGACATTCTTTTTCGCCATCGGCGTGTTCATCCTGCTGTACGTGGTGGCA
AGGATCGTGATCGCCGTGCGGTACAGATATCAGGGCAGCAACAATAAGAG
AATCTATAACGATATCGAGATGTCCAGGTTCCGCAAGTGA
Non-limiting example of canine CD86-2A-anti-
CD3scFv-2A-CD137L-2A-COCVG (polynucleotide
sequence):
(SEQ ID NO: 6)
ATGTACCTGCGCTGCACCATGGAGCTGAACAATATCCTGTTCGTGATGAC
ACTGCTGCTGTACGGCGCCGCCTCTATGAAGAGCCAGGCCTACTTCAACA
AGACCGGCGAGCTGCCCTGTCACTTCACAAACTCCCAGAATATCTCTCTG
GACGAGCTGGTGGTGTTTTGGCAGGACCAGGATAAGCTGGTGCTGTACGA
GCTGTATCGCGGCAAGGAGAACCCTCAGAATGTGCACAGAAAGTACAAGG
GCAGGACCTCCTTTGACAAGGATAACTGGACACTGCGGCTGCACAATATC
CAGATCAAGGATAAGGGCCTGTATCAGTGCTTCGTGCACCACAAGGGCCC
AAAGGGCCTGGTGCCCATGCACCAGATGAACTCCGACCTGTCTGTGCTGG
CCAACTTCAGCCAGCCCGAGATCATGGTGACCTCCAACCGGACAGAGAAC
AGCGGCATCATCAACCTGACCTGTAGCTCCATCCAGGGCTACCCCGAGCC
TAAGGAGATGTATTTCCTGGTGAAGACAGAGAACAGCAGCACCAAGTACG
ATACAGTGATGAAGAAGAGCCAGAACAATGTGACCGAGCTGTATAACGTG
TCTATCAGCCTGTCCTTTTCTGTGCCTGAGGCCAGCAACGTGAGCATCTT
CTGCGTGCTGCAGCTGGAGTCTATGAAGCTGCCCAGCCTGCCTTACAACA
TCGACGCCCACACCAAGCCAACACCCGACGGCGATCACATCCTGTGGATC
GCCGCCCTGCTGGTCATGCTGGTCATCCTGTGCGGCATGGTGTTCTTTCT
GACCCTGAGAAAGCGGAAGAAGAAGCAGCCTGGCCCATCCCACGAGTGTG
AGACAAATAAGGTGGAGCGCAAGGAGTCTGAGCAGACCAAGGAGCGCGTG
CGGTACCACGAGACAGAGCGGTCCGATGAGGCCCAGTGCGTGAACATCAG
CAAGACCGCCTCCGGCGACAATTCTACCACACAGTTCGGCAGCGGAGAGG
GAAGAGGCTCCCTGCTGACCTGTGGCGATGTGGAGGAGAACCCAGGACCT
GCCCTGCCAGTGACAGCCCTGCTGCTGCCTCTGGCCCTGCTGCTGCACGC
CGCCAGGCCAGAGGTGCAGCTGGTGGAGAGCGGAGGAGGACTGGTGCAGC
CCAAGGGCAGCCTGAAGCTGTCCTGCGCCACCTCTGGCTTTACATTCAAC
ATCTACGCCATGAATTGGGTGAGACAGGCACCAGGCAAGGGACTGGAGTG
GGTGGCCAGAATCAGGAGCAAGTCCAAGAATTATGCCACCTACTATGCCG
GCAGCGTGAAGGACCGCTTTACAATCTCCCGGGACGATTCTCAGAGCATG
CTGTACCTGCAGATGAACAATCTGAAGACCGAGGATACAGCCATGTACTA
TTGCGTGCGGAGAGGCTATTTCGACGTGTGGGGAGCAGGAACCACAGTGA
CCGTGTCCTCTAGCGGAGGAGGAGGCTCCGGAGGAGGAGGCTCTGGCGGC
GGCGGCAGCATCATCGTGATGACCCAGTCCCCAAAGTCCATGTCTATGAG
CGTGGGAGAGAGGGTGACACTGAGCTGTCGGGCCTCCGAGAACGTGGATT
CTTTTGTGAGCTGGTACCAGCAGAAGCCTGACCAGAGCCCACAGCTGCTG
ATCTTCGGCGCCTCCAATAGATCTACCGGCGTGCCCGATAGGTTTACCGG
CTCCGGCTCTGCCACAGACTTCACACTGACCATCACAAGCGTGCAGTCCG
AGGATCTGGCAGCATACCACTGCGGACAGACCTACTCCTATCCTTTTACC
TTCGGCTCTGGCACAAACCTGGAGATCAAGCGGAGCGACCCCACCACAAC
CCCAGCCCCAAGGCCACCTACCCCTGCCCCAACAATCGCCTCTCAGCCTC
TGAGCCTGAGACCAGAGGCCTGTAGGCCAGCCGCAGGGGGGGCAGTGCAC
ACCCGCGGCCTGGACTTCGCCTGCGATATCTACATCTGGGCACCCCTGGC
CGGAACCTGTGGCGTGCTGCTGCTGAGCCTGGTCATCACCCTGTATTGCA
ACCACCGGAATAGGCGCCGGGTGTGCAAGTGTCCAAGACCTGTGGTGGGC
TCTGGAGAGGGAAGGGGCAGCCTGCTGACTTGCGGCGATGTGGAAGAAAA
CCCAGGACCAATGAGGCCACGGAGCGACGCCGCCCCTGATCCAGAGGCAC
CAAGGCCACCCGCCCCTCCAGGCAGAGCCTGCAGCCCACTGCCTTGGGCA
CTGTCCGCCGCAATGCTGCTGCTGGTGGGCACCTGCGCCGCCTGTGCCCT
GCGCGCCTGGGTGGTGCCAGGACCAAGGCCCCCTGCCCTGCCCGCCCTGC
CTGCCCCACTGCCCGATGCCGGCGCCAGGCTGCCAGACAGCCCACAGGCC
GTGTTCGCACAGCTGGTGGCCAGAGATGTGCAGCTGAAGGAGGGACCACT
GAGGTGGTACAGCGACCCCGGCCTGGCCGGCGTGTTTCTGGGACCCGGCC
TGTCCTATGACCAGCACACAAGAGAGCTGATGGTGGTGGAGCCTGGCCTG
TACTACGTGTTCCTGCACCTGAAGCTGCAGAGAGTGATGTCCTCTACCGG
CAGCGGCTCCGTGTCTGCCGCCCTGCACCTGCAGCCACTGGGAACCGAGG
CAGCCGCCCTGGACCTGACACTGGATCTGCCACCCCCTAGCTCCGAGGCC
AGAGATTCTGCCGCAGGCTTTAGGGGCAGCCTGCTGCACCTGGACGCAGG
ACAGAGACTGAGGGTGCACCTGAGGGCAGAGGCAGGAGCACACCCTGCAT
GGCAGCTGGCCCAGGGAGCAACAATCCTGGGACTGTTCCGGGTGGCAACC
AAGGTGCCAACAGGACTGCCTTCTAGCTGGCCAATGGATACCGGACCAGG
CTCCCCACCACTGGACGGAGAGGGCTCCGGCGAGGGCAGAGGCTCTCTGC
TGACCTGCGGCGACGTGGAGGAGAACCCTGGACCAAATTTTCTGCTGCTG
ACATTCATCGTGCTGCCTCTGTGCTCCCACGCCAAGTTTTCTATCGTGTT
CCCACAGAGCCAGAAGGGCAACTGGAAGAATGTGCCTTCCTCTTACCACT
ATTGCCCAAGCTCCTCTGACCAGAACTGGCACAATGATCTGCTGGGCATC
ACCATGAAGGTGAAGATGCCTAAGACACACAAGGCCATCCAGGCAGATGG
ATGGATGTGCCACGCAGCCAAGTGGATCACAACCTGTGACTTTAGGTGGT
ACGGCCCCAAGTATATCACCCACTCTATCCACAGCATCCAGCCTACAAGC
GAGCAGTGCAAGGAGTCCATCAAGCAGACCAAGCAGGGCACATGGATGTC
TCCAGGCTTCCCTCCACAGAACTGTGGCTACGCCACCGTGACAGACAGCG
TGGCAGTGGTGGTGCAGGCAACCCCTCACCACGTGCTGGTGGATGAGTAT
ACAGGCGAGTGGATCGACAGCCAGTTTCCCAACGGCAAGTGCGAGACCGA
GGAGTGTGAGACAGTGCACAATAGCACCGTGTGGTACTCCGATTATAAGG
TGACCGGCCTGTGCGACGCCACACTGGTGGATACCGAGATCACATTCTTT
TCCGAGGACGGCAAGAAGGAGTCTATCGGCAAGCCCAACACCGGCTACAG
ATCCAATTACTTCGCCTATGAGAAGGGCGATAAGGTGTGCAAGATGAATT
ATTGTAAGCACGCCGGGGTGCGGCTGCCAAGCGGCGTGTGGTTTGAGTTC
GTGGACCAGGACGTGTACGCAGCAGCAAAGCTGCCTGAGTGCCCAGTGGG
AGCAACCATCTCCGCCCCCACCCAGACATCCGTGGACGTGTCTCTGATCC
TGGATGTGGAGCGCATCCTGGACTACAGCCTGTGCCAGGAGACCTGGAGC
AAGATCCGGTCCAAGCAGCCCGTGTCTCCTGTGGACCTGAGCTATCTGGC
ACCAAAGAACCCAGGAACAGGACCTGCCTTTACCATCATCAATGGCACAC
TGAAGTACTTCGAGACCCGCTATATCCGGATCGACATCGATAACCCAATC
ATCAGCAAGATGGTGGGCAAGATCAGCGGCTCCCAGACCGAGAGAGAGCT
GTGGACAGAGTGGTTCCCCTACGAGGGCGTGGAGATCGGCCCTAATGGCA
TCCTGAAGACCCCTACAGGCTATAAGTTTCCACTGTTCATGATCGGCCAC
GGCATGCTGGACTCTGATCTGCACAAGACCAGCCAGGCCGAGGTGTTTGA
GCACCCACACCTGGCAGAGGCACCAAAGCAGCTGCCTGAGGAGGAGACCC
TGTTCTTTGGCGATACAGGCATCTCCAAGAACCCTGTGGAGCTGATCGAG
GGCTGGTTTAGCTCCTGGAAGTCTACCGTGGTGACATTCTTTTTCGCCAT
CGGCGTGTTCATCCTGCTGTACGTGGTGGCCAGAATCGTGATCGCCGTGC
GCTACCGGTATCAGGGCTCTAACAATAAGAGGATCTATAATGACATCGAG
ATGAGCAGATTCAGGAAGTGA
Non-limiting example of human CD86-2A-anti-
CD3scFv-2A-CD137L-2A-COCVG (polypeptide sequence):
(SEQ ID NO: 7)
MDPQCTMGLSNILFVMAFLLSGAAPLKIQAYFNETADLPCQFANSQNQSL
SELVVFWQDQENLVLNEVYLGKEKFDSVHSKYMGRTSFDSDSWTLRLHNL
QIKDKGLYQCIIHHKKPTGMIRIHQMNSELSVLANFSQPEIVPISNITEN
VYINLTCSSIHGYPEPKKMSVLLRTKNSTIEYDGIMQKSQDNVTELYDVS
ISLSVSFPDVTSNMTIFCILETDKTRLLSSPFSIELEDPQPPPDHIPWIT
AVLPTVIICVMVFCLILWKWKKKKRPRNSYKCGTNTMEREESEQTKKREK
IHIPERSDEAQRVEKSSKTSSCDKSDTCFGSGEGRGSLLTCGDVEENPGP
ALPVTALLLPLALLLHAARPQVQLLESGAELARPGASVKMSCKASGYTFT
RYTMHWVKQRPGQGLEWIGYINPSRGYTNYNQKFKDKATLTTDKSSSTAY
MQLSSLTSEDSAVYYCAGYYDDHYCLDYWGQGTLVTVSSGGGGSGGGGSG
GGGSDIVMTQSPAIMSASPGEKVTMTCSASSSVSYMNWYQQKSGTSPKRW
IYDTSKLASGVPAHFRGSGSGTSYSLTISGMEAEDAATYYCQQWSSNPFT
FGSGTKLEIKRDPSTTTPAPRPPTPAPTIASQPLSLRPEACRPAAGGAVH
TRGLDFACDIYIWAPLAGTCGVLLLSLVITLYCKRGRKKGSGEGRGSLLT
CGDVEENPGPMEYASDASLDPEAPWPPAPRARACRVLPWALVAGLLLLLL
LAAACAVFLACPWAVSGARASPGSAASPRLREGPELSPDDPAGLLDLRQG
MFAQLVAQNVLLIDGPLSWYSDPGLAGVSLTGGLSYKEDTKELVVAKAGV
YYVFFQLELRRVVAGEGSGSVSLALHLQPLRSAAGAAALALTVDLPPASS
EARNSAFGFQGRLLHLSAGQRLGVHLHTEARARHAWQLTQGATVLGLFRV
TPEIPAGLPSPRSEGSGEGRGSLLTCGDVEENPGPNFLLLTFIVLPLCSH
AKFSIVFPQSQKGNWKNVPSSYHYCPSSSDQNWHNDLLGITMKVKMPKTH
KAIQADGWMCHAAKWITTCDFRWYGPKYITHSIHSIQPTSEQCKESIKQT
KQGTWMSPGFPPQNCGYATVTDSVAVVVQATPHHVLVDEYTGEWIDSQFP
NGKCETEECETVHNSTVWYSDYKVTGLCDATLVDTEITFFSEDGKKESIG
KPNTGYRSNYFAYEKGDKVCKMNYCKHAGVRLPSGVWFEFVDQDVYAAAK
LPECPVGATISAPTQTSVDVSLILDVERILDYSLCQETWSKIRSKQPVSP
VDLSYLAPKNPGTGPAFTIINGTLKYFETRYIRIDIDNPIISKMVGKISG
SQTERELWTEWFPYEGVEIGPNGILKTPTGYKFPLFMIGHGMLDSDLHKT
SQAEVFEHPHLAEAPKQLPEEETLFFGDTGISKNPVELIEGWFSSWKSTV
VTFFFAIGVFILLYVVARIVIAVRYRYQGSNNKRIYNDIEMSRFRK
Non-limiting example of murine CD86-2A-anti-
CD3scFv-2A-CD137L-2A-COCVG (polypeptide sequence):
(SEQ ID NO: 8)
MDPRCTMGLAILIFVTVLLISDAVSVETQAYFNGTAYLPCPFTKAQNISL
SELVVFWQDQQKLVLYEHYLGTEKLDSVNAKYLGRTSFDRNNWTLRLHNV
QIKDMGSYDCFIQKKPPTGSIILQQTLTELSVIANFSEPEIKLAQNVTGN
SGINLTCTSKQGHPKPKKMYFLITNSTNEYGDNMQISQDNVTELFSISNS
LSLSFPDGVWHMTVVCVLETESMKISSKPLNFTQEFPSPQTYWKEITASV
TVALLLVMLLIIVCHKKPNQPSRPSNTASKLERDSNADRETINLKELEPQ
IASAKPNAEGSGEGRGSLLTCGDVEENPGPALPVTALLLPLALLLHAARP
EVQLVESGGGLVQPGKSLKLSCEASGFTFSGYGMHWVRQAPGRGLESVAY
ITSSSINIKYADAVKGRFTVSRDNAKNLLFLQMNILKSEDTAMYYCARFD
WDKNYWGQGTMVTVSSGGGGSGGGGSGGGGSDIQMTQSPSSLPASLGDRV
TINCQASQDISNYLNWYQQKPGKAPKLLIYYTNKLADGVPSRFSGSGSGR
DSSFTISSLESEDIGSYYCQQYYNYPWTFGPGTKLEIKRDPSTTTPAPRP
PTPAPTIASQPLSLRPEACRPAAGGAVHTRGLDFACDIYIWAPLAGTCGV
LLLSLVITLYCKRGRKKGSGEGRGSLLTCGDVEENPGPMDQHTLDVEDTA
DARHPAGTSCPSDAALLRDTGLLADAALLSDTVRPTNAALPTDAAYPAVN
VRDREAAWPPALNFCSRHPKLYGLVALVLLLLIAACVPIFTRTEPRPALT
ITTSPNLGTRENNADQVTPVSHIGCPNTTQQGSPVFAKLLAKNQASLCNT
TLNWHSQDGAGSSYLSQGLRYEEDKKELVVDSPGLYYVFLELKLSPTFTN
TGHKVQGWVSLVLQAKPQVDDFDNLALTVELFPCSMENKLVDRSWSQLLL
LKAGHRLSVGLRAYLHGAQDAYRDWELSYPNTTSFGLFLVKPDNPWEGSG
EGRGSLLTCGDVEENPGPNFLLLTFIVLPLCSHAKFSIVFPQSQKGNWKN
VPSSYHYCPSSSDQNWHNDLLGITMKVKMPKTHKAIQADGWMCHAAKWIT
TCDFRWYGPKYITHSIHSIQPTSEQCKESIKQTKQGTWMSPGFPPQNCGY
ATVTDSVAVVVQATPHHVLVDEYTGEWIDSQFPNGKCETEECETVHNSTV
WYSDYKVTGLCDATLVDTEITFFSEDGKKESIGKPNTGYRSNYFAYEKGD
KVCKMNYCKHAGVRLPSGVWFEFVDQDVYAAAKLPECPVGATISAPTQTS
VDVSLILDVERILDYSLCQETWSKIRSKQPVSPVDLSYLAPKNPGTGPAF
TIINGTLKYFETRYIRIDIDNPIISKMVGKISGSQTERELWTEWFPYEGV
EIGPNGILKTPTGYKFPLFMIGHGMLDSDLHKTSQAEVFEHPHLAEAPKQ
LPEEETLFFGDTGISKNPVELIEGWFSSWKSTVVTFFFAIGVFILLYVVA
RIVIAVRYRYQGSNNKRIYNDIEMSRFRK
Non-limiting example of canine CD86-2A-anti-
CD3scFv-2A-CD137L-2A-COCVG (polypeptide sequence):
(SEQ ID NO: 9)
MYLRCTMELNNILFVMTLLLYGAASMKSQAYFNKTGELPCHFTNSQNISL
DELVVFWQDQDKLVLYELYRGKENPQNVHRKYKGRTSFDKDNWTLRLHNI
QIKDKGLYQCFVHHKGPKGLVPMHQMNSDLSVLANFSQPEIMVTSNRTEN
SGIINLTCSSIQGYPEPKEMYFLVKTENSSTKYDTVMKKSQNNVTELYNV
SISLSFSVPEASNVSIFCVLQLESMKLPSLPYNIDAHTKPTPDGDHILWI
AALLVMLVILCGMVFFLTLRKRKKKQPGPSHECETNKVERKESEQTKERV
RYHETERSDEAQCVNISKTASGDNSTTQFGSGEGRGSLLTCGDVEENPGP
ALPVTALLLPLALLLHAARPEVQLVESGGGLVQPKGSLKLSCATSGFTFN
IYAMNWVRQAPGKGLEWVARIRSKSKNYATYYAGSVKDRFTISRDDSQSM
LYLQMNNLKTEDTAMYYCVRRGYFDVWGAGTTVTVSSSGGGGSGGGGSGG
GGSIIVMTQSPKSMSMSVGERVTLSCRASENVDSFVSWYQQKPDQSPQLL
IFGASNRSTGVPDRFTGSGSATDFTLTITSVQSEDLAAYHCGQTYSYPFT
FGSGTNLEIKRSDPTTTPAPRPPTPAPTIASQPLSLRPEACRPAAGGAVH
TRGLDFACDIYIWAPLAGTCGVLLLSLVITLYCNHRNRRRVCKCPRPVVG
SGEGRGSLLTCGDVEENPGPMRPRSDAAPDPEAPRPPAPPGRACSPLPWA
LSAAMLLLVGTCAACALRAWVVPGPRPPALPALPAPLPDAGARLPDSPQA
VFAQLVARDVQLKEGPLRWYSDPGLAGVFLGPGLSYDQHTRELMVVEPGL
YYVFLHLKLQRVMSSTGSGSVSAALHLQPLGTEAAALDLTLDLPPPSSEA
RDSAAGFRGSLLHLDAGQRLRVHLRAEAGAHPAWQLAQGATILGLFRVAT
KVPTGLPSSWPMDTGPGSPPLDGEGSGEGRGSLLTCGDVEENPGPNFLLL
TFIVLPLCSHAKFSIVFPQSQKGNWKNVPSSYHYCPSSSDQNWHNDLLGI
TMKVKMPKTHKAIQADGWMCHAAKWITTCDFRWYGPKYITHSIHSIQPTS
EQCKESIKQTKQGTWMSPGFPPQNCGYATVTDSVAVVVQATPHHVLVDEY
TGEWIDSQFPNGKCETEECETVHNSTVWYSDYKVTGLCDATLVDTEITFF
SEDGKKESIGKPNTGYRSNYFAYEKGDKVCKMNYCKHAGVRLPSGVWFEF
VDQDVYAAAKLPECPVGATISAPTQTSVDVSLILDVERILDYSLCQETWS
KIRSKQPVSPVDLSYLAPKNPGTGPAFTIINGTLKYFETRYIRIDIDNPI
ISKMVGKISGSQTERELWTEWFPYEGVEIGPNGILKTPTGYKFPLFMIGH
GMLDSDLHKTSQAEVFEHPHLAEAPKQLPEEETLFFGDTGISKNPVELIE
GWFSSWKSTVVTFFFAIGVFILLYVVARIVIAVRYRYQGSNNKRIYNDIE
MSRFRK

In some embodiments, the protein sequence of the CD86 shares at least 75%, 80%, 85%, 90%, 95%, 99%, or 100% identity with SEQ ID NO: 10.

(SEQ ID NO: 10)
MDPQCTMGLSNILFVMAFLLSGAAPLKIQAYFNETADLPCQFANSQNQSL
SELVVFWQDQENLVLNEVYLGKEKFDSVHSKYMGRTSFDSDSWTLRLHNL
QIKDKGLYQCIIHHKKPTGMIRIHQMNSELSVLANFSQPEIVPISNITEN
VYINLTCSSIHGYPEPKKMSVLLRTKNSTIEYDGIMQKSQDNVTELYDVS
ISLSVSFPDVTSNMTIFCILETDKTRLLSSPFSIELEDPQPPPDHIPWIT
AVLPTVIICVMVFCLILWKWKKKKRPRNSYKCGTNTMEREESEQTKKREK
IHIPERSDEAQRVEKSSKTSSCDKSDTCF

In some embodiments, the protein sequence of the anti-CD3scFv shares at least 75%, 80%, 85%, 90%, 95%, 99%, or 100% identity with SEQ ID NO: 11. Unless otherwise indicated, “anti-CD3scFv” refers to a single chain variable fragment capable of specifically binding to CD3 fused to a transmembrane domain capable of tethering the anti-CD3scFv to the lentiviral particle.

(SEQ ID NO: 11)
ALPVTALLLPLALLLHAARPQVQLLESGAELARPGASVKMSCKASGYTFT
RYTMHWVKQRPGQGLEWIGYINPSRGYTNYNQKFKDKATLTTDKSSSTAY
MQLSSLTSEDSAVYYCAGYYDDHYCLDYWGQGTLVTVSSGGGGSGGGGSG
GGGSDIVMTQSPAIMSASPGEKVTMTCSASSSVSYMNWYQQKSGTSPKRW
IYDTSKLASGVPAHFRGSGSGTSYSLTISGMEAEDAATYYCQQWSSNPFT
FGSGTKLEIKRDPSTTTPAPRPPTPAPTIASQPLSLRPEACRPAAGGAVH
TRGLDFACDIYIWAPLAGTCGVLLLSLVITLYCKRGRKK

In some embodiments, the protein sequence of the CD137L shares at least 75%, 80%, 85%, 90%, 95%, 99%, or 100% identity with SEQ ID NO: 12.

(SEQ ID NO: 12)
MEYASDASLDPEAPWPPAPRARACRVLPWALVAGLLLLLLLAAACAVFLA
CPWAVSGARASPGSAASPRLREGPELSPDDPAGLLDLRQGMFAQLVAQNV
LLIDGPLSWYSDPGLAGVSLTGGLSYKEDTKELVVAKAGVYYVFFQLELR
RVVAGEGSGSVSLALHLQPLRSAAGAAALALTVDLPPASSEARNSAFGFQ
GRLLHLSAGQRLGVHLHTEARARHAWQLTQGATVLGLFRVTPEIPAGLPS
PRSE

In some embodiments, the protein sequence of the COCVG shares at least 75%, 80%, 85%, 90%, 95%, 99%, or 100% identity with SEQ ID NO: 13.

(SEQ ID NO: 13)
NFLLLTFIVLPLCSHAKFSIVFPQSQKGNWKNVPSSYHYCPSSSDQNWHN
DLLGITMKVKMPKTHKAIQADGWMCHAAKWITTCDFRWYGPKYITHSIHS
IQPTSEQCKESIKQTKQGTWMSPGFPPQNCGYATVTDSVAVVVQATPHHV
LVDEYTGEWIDSQFPNGKCETEECETVHNSTVWYSDYKVTGLCDATLVDT
EITFFSEDGKKESIGKPNTGYRSNYFAYEKGDKVCKMNYCKHAGVRLPSG
VWFEFVDQDVYAAAKLPECPVGATISAPTQTSVDVSLILDVERILDYSLC
QETWSKIRSKQPVSPVDLSYLAPKNPGTGPAFTIINGTLKYFETRYIRID
IDNPIISKMVGKISGSQTERELWTEWFPYEGVEIGPNGILKTPTGYKFPL
FMIGHGMLDSDLHKTSQAEVFEHPHLAEAPKQLPEEETLFFGDTGISKNP
VELIEGWFSSWKSTVVTFFFAIGVFILLYVVARIVIAVRYRYQGSNNKRI
YNDIEMSRFRK

### 1.3 Promoters and Gene Control Elements

The present disclosure contemplates multicistronic vectors comprising a polynucleotide operatively linked to a promoter. In some embodiments the polynucleotide is operatively linked to an enhancer. In some embodiments, a “strong” promoter is used. The strength of a promoter is determined in part by the attributes of the cell in which it operates. In some embodiments, the strong promoter of the present disclosure results in high-level expression of gene elements to which it is operatively linked in a target cell, such as a TIL. Strong promoters include, without limitation, cytomegalovirus (CMV) and murine stem cell virus (MSCV), phosphoglycerate kinase (PGK), a promoter sequence comprised of the CMV enhancer and portions of the chicken beta-actin promoter and the rabbit beta-globin gene (CAG), a promoter sequence comprised of portions of the SV40 promoter and CD43 promoter (SV40/CD43), and a synthetic promoter that contains the U3 region of a modified MoMuLV LTR with myeloproliferative sarcoma virus enhancer (MND). Exemplary strong promoters useful in the compositions and method of the present disclosure are provided by Jones et al. Lentiviral vector design for optimal T cell receptor gene expression in the transduction of peripheral blood lymphocytes and tumor-infiltrating lymphocytes. Hum Gene Ther. 2009 June; 20(6):630-40. In some embodiments, the strong promoter may be a synthetic strong promoter. Exemplary synthetic strong promoters are provided by Schlabach et al. Proc. Nat'l Acad. Sci. USA. 2010 Feb. 9; 107(6): 2538-2543. In some embodiments, other promoters are used. In some embodiments, any promoter active in the packaging cell line is used. In some embodiments, an inducible promoter is used, e.g. a drug-inducible promoter.

In some embodiments, vectors of the present disclosure may comprise the Woodchuck Hepatitis Virus Posttranscriptional Regulatory Element (wPRE) or a nucleic acid sequence substantially identical to wPRE. See U.S. Pat. No. 6,136,597; Lee et al. Optimizing regulatable gene expression using adenoviral vectors. Exp Physiol. 90 (1): 33-7 (2005). Variants of the wPRE element with reduced size are known in the art. wPRE-O refers to a variant of wPRE with the intermediate size.

### 1.4 Linkers

In some embodiments, multicistronic vectors of the present disclosure comprise a polynucleotide sequence encoding a plurality of polypeptides joined by linkers comprising peptides capable of inducing ribosome skipping or self-cleavage. In some embodiments, the linker comprises a 2A peptide. The term “2A peptide” as used herein refers to a class of ribosome skipping or self-cleaving peptide configured to generate two or more proteins from a single open reading frame. 2A peptides are 18-22 residue-long viral oligopeptides that mediate “cleavage” of polypeptides during translation in eukaryotic cells. “2A peptide” may refer to peptides with various amino acid sequences. In the present disclosure it will be understood that where a lentiviral vector comprises two or more 2A peptides, the 2A peptides may be identical to one another or different. Detailed methodology for design and use of 2A peptides is provided by Szymczak-Workman et al. Cold Spring Harb. Protoc. (2012) 2012(2):199-204. In the literature, 2A peptides are often referred to as self-cleaving peptides, but mechanistic studies have shown that the “self-cleavage” observed is actually a consequence of the ribosome's skipping the formation of the glycyl-prolyl peptide bond at the C terminus of the 2A peptide. Donnelly et al. (2001) J Gen Virol. 282(5):1027-41. The present invention is not bound by theory or limited to any particular mechanistic understanding of 2A peptide function.

Exemplary 2A peptides include, without limitation, those listed in Table 1.

TABLE 1

| | Source | Nucleotide | Peptide |
|---|---|---|---|
| P2A | porcine | GCT ACT AAC TTC AGC | ATNFSLLKQAGDVEENPGP |
| | teschovirus-1 | CTG CTG AAG CAG GCT | (SEQ ID NO: 14) |
| | | GGA GAC GTG GAG GAG | |
| | | AAC CCT GGA CCT | |
| | | (SEQ ID NO: 18) | |
| T2A | Thoseaasigna virus | GAG GGC AGA GGA AGT | EGRGSLLTCGDVEENPGP |
| | | CTG CTA ACA TGC GGT | (SEQ ID NO: 15) |
| | | GAC GTC GAG GAG AAT | |
| | | CCT GGA CCT | |
| | | (SEQ ID NO: 19) | |
| E2A | equine rhinitis A | CAG TGT ACT AAT TAT | QCTNYALLKLAGDCESNPGP |
| | virus (ERAV) | GCT CTC TTG AAA TTG | (SEQ ID NO: 16) |
| | | GCT GGA GAT GTT GAG | |
| | | AGC AAC CCT GGA CCT | |
| | | (SEQ ID NO: 20) | |
| F2A | foot-and-mouth | GTG AAA CAG ACT TTG | VKQTLNFDLLKLAGDVESNPGP |
| | disease virus | AAT TTT GAC CTT CTC | (SEQ ID NO: 17) |
| | (FMDV) | AAG TTG GCG GGA GAC | |
| | | GTG GAG TCC AAC CCT | |
| | | GGA CCT | |
| | | (SEQ ID NO: 21) | |

Optionally, one or more of the linkers further comprises a sequence encoding the residues Gly-Ser-Gly, which is in some embodiments N-terminal to the 2A peptide. N-terminal to the 2A peptide means that the sequence encoding the residues is upstream to the sequence encoding the 2A peptide. Generally, the Gly-Ser-Gly motif will be immediately N-terminal to the 2A peptide or between 1 to 10 other amino acid residues are inserted between the motif and the 2A peptide. In some embodiments, the polynucleotide sequence encoding this motif is GGA AGC GGA. As with any peptide-encoding polynucleotide, the nucleotide sequence may be altered without changing the encoded peptide sequence. Substitution of amino acid residues is within the skill of those in the art, and it will be understood that the term 2A peptide refers to variants of the foregoing that retain the desired skipping/self-cleavage activity but, optionally, have 1, 2, 3, 4, 5, 6, 7, 8, 9, 10 or more substitutions relative to the reference 2A peptide sequence. Exemplary 2A peptides are described in Kim et al. PLOS ONE 6(4): e18556. In some embodiments, two or more different 2A peptides are used in the same construct. Varied 2A peptides have been reported to result in improved expression. See Liu et al. Sci Rep. 2017; 7:2193.

### 1.5 Fusion Glycoproteins

Various fusion glycoproteins can be used to pseudotype lentiviral vecotrs. While the most commonly used example is the envelope glycoprotein from vesicular stomatitis virus (VSVG), many other viral proteins have also been used for pseudotyping of lentiviral vectors. See Joglekar et al. Human Gene Therapy Methods 28:291-301 (2017). The present disclosure contemplates substitution of various fusion glycoproteins. Notably, some fusion glycoproteins result in higher vector efficiency.

In some embodiments, pseudotyping a fusion glycoprotein or functional variant thereof facilitates targeted transduction of specific cell types, including, but not limited to, T cells or NK-cells. In some embodiments, the fusion glycoprotein or functional variant thereof is/are full-length polypeptide(s), functional fragment(s), homolog(s), or functional variant(s) of Human immunodeficiency virus (HIV) gp160, Murine leukemia virus (MLV) gp70, Gibbon ape leukemia virus (GALV) gp70, Feline leukemia virus (RD114) gp70, Amphotropic retrovirus (Ampho) gp70, 10A1 MLV (10A1) gp70, Ecotropic retrovirus (Eco) gp70, Baboon ape leukemia virus (BaEV) gp70, Measles virus (MV) H and F, Nipah virus (NiV) H and F, Rabies virus (RabV) G, Mokola virus (MOKV) G, Ebola Zaire virus (EboZ) G, Lymphocytic choriomeningitis virus (LCMV) GP1 and GP2, Baculovirus GP64, Chikungunya virus (CHIKV) E1 and E2, Ross River virus (RRV) E1 and E2, Semliki Forest virus (SFV) E1 and E2, Sindbis virus (SV) E1 and E2, Venezualan equine encephalitis virus (VEEV) E1 and E2, Western equine encephalitis virus (WEEV) E1 and E2, Influenza A, B, C, or D HA, Fowl Plague Virus (FPV) HA, Vesicular stomatitis virus VSV-G, or Chandipura virus and Piry virus CNV-G and PRV-G.

In some embodiments, the fusion glycoprotein or functional variant thereof is a full-length polypeptide, functional fragment, homolog, or functional variant of the G protein of Vesicular Stomatitis Alagoas Virus (VSAV), Carajas Vesiculovirus (CJSV), Chandipura Vesiculovirus (CHPV), Cocal Vesiculovirus (COCV), Vesicular Stomatitis Indiana Virus (VSIV), Isfahan Vesiculovirus (ISFV), Maraba Vesiculovirus (MARAV), Vesicular Stomatitis New Jersey virus (VSNJV), Bas-Congo Virus (BASV). In some embodiments, the fusion glycoprotein or functional variant thereof is the Cocal virus G protein.

### 1.6 Checkpoint-Inhibiting Ligands

In some embodiments, the lentiviral vector systems of the present disclosure further comprise a nucleic acid sequence encoding a checkpoint-inhibiting ligand. Optionally, the checkpoint-inhibiting ligand is capable of blocking the PD-1/PD-L1 checkpoint. Optionally, the checkpoint-inhibiting ligand is capable of blocking the Tim-3 checkpoint.

Checkpoint inhibitor therapy is a form of cancer treatment that uses agents to stimulate or inhibit immune checkpoints and thereby modulate the immune response. Tumors may use checkpoints to protect themselves from the immune system of the subject or from therapeutic agents used in cancer immunotherapy. The present disclosure provides a lentiviral vector system comprising a nucleic acid sequence encoding a checkpoint-inhibiting ligand, wherein lentiviral particles produced from the lentiviral vector system display the checkpoint-inhibiting ligand on their surface, and therefore administration of the lentiviral particle results in delivery of the checkpoint-inhibiting ligand to the subject at the site of therapeutic use. The present disclosure further provides lentiviral vector systems comprising a nucleic acid sequence encoding a checkpoint-inhibiting ligand, whereby administration of lentiviral particles produced from the lentiviral vector system delivers the polynucleotide sequence to target cells, which then express the checkpoint-inhibiting ligand at the site of therapeutic use.

Examples of checkpoint-inhibitor ligands provided by the present disclosure include, without limitation, anti-CTLA-4 antibody, anti-PD-1 antibodies, and anti-PD-L1 antibodies or any non-antibody ligands (e.g. nanobodies, DARPins) that interact with CTLA4, PD-1, or PD-L1, respectively. In some embodiments, the checkpoint-inhibiting ligand is capable of blocking the PD-1/PD-L1 checkpoint and/or the Tim-3 checkpoint and/or the CTLA-4 checkpoint. Use of checkpoint inhibition is reviewed in, e.g., Anderson et al. Tim-3: an emerging target in the cancer immunotherapy landscape. Cancer Immunol Res. 2014 May; 2(5):393-8.

### 1.6 Resistance to Immunosuppressive Drugs

In some embodiments, the lentiviral vector system of the present disclosure further comprises a nucleic acid sequence (e.g., on the transfer plasmid) that provides resistance to an immunosuppressive drug. A nucleic acid sequence that provides resistance to a immunosuppressive drug will, in some embodiments, facilitate selective expansion of target cells when the immunosuppressive drug is administered to a patient during any of the methods for treating a subject or any of the methods for expanding T-cells capable of recognizing and killing tumor cells in a subject in need thereof provided by the present disclosure. In some embodiments, the immunosuppressive drug is methotrexate rapamycin, a rapalog, tacrolimus, cyclosporine, or a combination thereof.

In some embodiments, the lentiviral particle facilitates selective expansion of target cells by conferring resistance to an immunosuppressive drug to transduced cells. The present disclosure provides a lentiviral vector system that comprises any of the nucleic sequences that confer resistance to an immunosuppressive drug known in the art. Examples of immunosuppressive drugs include, without limitation, rapamycin or a derivative thereof, a rapalog or a derivative thereof, tacrolimus or a derivative thereof, cyclosporine or a derivative thereof, methotrexate or a derivative thereof, and mycophenolate mofetil (MMF) or a derivative thereof. Various resistance genes are known in the art. Resistance to rapamycin may be conferred by a polynucleotide sequence encoding the protein domain FRb, found in the mTOR domain and known to be the target of the FKBP-rapamycin complex. Resistance to tacrolimus may be conferred by a polynucleotide sequence encoding the calcineurin mutant CNa22 or calcineurin mutant CNb30. Resistance to cyclosporine may be conferred by a polynucleotide sequence encoding the calcineurin mutant CNa12 or calcineurin mutant CNb30. These calcineurin mutants are described in Brewin et al. (2009) Blood. (23):4792-803. Resistance to methotrexate can be provided by various mutant forms of di-hydrofolate reducatse (DHFR) (see Volpato et al. (2011) J Mol Recognit 24:188-198), and resistance to MMF can be provided by various mutant forms of inosine monophosphate dehydrogenase (IMPDH) (Yam et al. (2006) Mol. Ther. 14: 236-244).

Immunosuppressive drugs are commonly used prior to, during, and/or after ACT. In some embodiments, use of an immunosuppressive drug may improve treatment outcomes. In some embodiments, use of an immunosuppressive drug may diminish side effects of treatment, such as, without limitation, acute graft-versus-host disease, chronic graft-versus-host disease, and post-transplant lymphoproliferative disease. The present disclosure contemplates use of immunosuppressive drugs with any of the methods of treating or preventing a disease or condition of the present disclosure, including, without limitation, methods of the present disclosure in which the lentiviral vector confers resistance to an immunosuppressive drug to transduced cells.

### 2 Packaging Cell Lines

In another aspect, the present disclosure provides packaging cell lines for generating lentiviral particles capable of activating and efficiently transducing T cells, comprising cultured cells capable of packaging a lentivirus vector, wherein the cultured cells are genetically engineered to express a T-cell activation or co-stimulation molecule, or are induced to transiently express a T-cell activation or co-stimulation molecule via transient transfection. The packaging cell lines of the present disclosure may be used with any lentiviral vector including but not limited to those previously described.

In some embodiments, the packaging cell line is a HEK-293T cell line. Similar results can be achieved with other cell lines, including, without limitation, HEK-293T cell lines engineered to be deficient in B2M or other immunologically active surface proteins. Other cell lines that are transfectable in vitro and capable of high titer lentiviral vector production can be used—e.g., cell lines that comprise the gene sequence for polyoma virus large T antigen operatively linked to a promoter.

The packaging cell line may be, in some embodiments, genetically modified to lack MHC class I expression, MHC class II expression, or expression of inhibitory checkpoint ligands, such as PD-L1 (a PD-1 ligand), or ligands for TIM3. As expression of inhibitory ligands by the packaging cell line could limit T-cell activation by the lentiviral particles, these genetic modification serve in some embodiments to eliminate such inhibitory signals, further promoting T-cell activation and transduction by the lentiviral particles.

In some embodiments, a packaging cell line is genetically engineered to comprise one or more genes useful in packing lentiviral vectors into lentiviral particles. In some embodiments, a packaging cell line may comprise polynucleotide sequences encoding the genes gag-pol, env, and rev. In a typical lentiviral vector of the present invention, at least part of one or more of the gag-pol and env protein coding regions may be removed from the lentiviral vector and provided by the packing cell line. Lentiviral vector systems may be packaged according to the methods provided in T. Dull et al, J Virol. 72:8463-71 (1998), which is incorporated herein in its entirety. Exemplary packaging cell lines are provide in Retroviruses Cold Spring Harbour Laboratory (Coffin et al., eds) (1997).

The present disclosure further provides for genetically engineering the packaging cell line to improve immunological attributes of the lentiviral vectors and particles of the present disclosure in other ways, including, without limitation, adding genes, deleting genes, and introducing point mutations into genes.

### 3 Non-Viral Proteins

In some embodiments, the multicistronic vectors of the disclosure comprise a polynucleotide operatively linked to a promoter, wherein the polynucleotide encodes a plurality of polypeptides joined by linkers comprising peptides capable of inducing ribosome skipping or self-cleavage, and wherein the plurality of polypeptides comprise a fusion glycoprotein or functional variant thereof and one or more non-viral proteins capable of viral surface display. Various non-viral proteins capable of viral surface display are provided by the present disclosure. In some embodiments the non-viral proteins are co-stimulatory molecules.

Conventionally, lentiviral transduction in vitro requires additional of an exogenous activating agent, such as a “stimbead,” for example Dynabeads™ Human T-Activator CD3/CD28. Lentiviral particles made using the multicistronic vectors of the present disclosure incorporate one or more copies of non-viral proteins encoded by the multicistronic vector into the lentiviral particle. Where the non-viral proteins are T-cell activation or co-stimulation molecule(s), the incorporation of T-cell activation or co-stimulation molecule(s) in the lentiviral particle renders the lentiviral particle capable of activating and efficiently transducing T cells in the absence of, or in the presence of lower amounts of, an exogenous activating agent, i.e. without a stimbead or equivalent agent. This permits the lentiviral particles made using these mutlicistronic vecotrs to be used in vivo, where exogenous delivery of an activating agent may be impractical.

In some embodiments, the T-cell activation or co-stimulation molecule may be selected from the group consisting of an anti-CD3 antibody, CD28 ligand (CD28L), and 41bb ligand (41BBL or CD137L). Various T-cell activation or co-stimulation molecules are known in the art and include, without limitation, agents that specifically bind any of the T-cell expressed proteins CD3, CD28, CD134 also known as OX40, or 41bb also known as 4-1BB or CD137 or TNFRSF9. For example, an agent that specifically binds CD3 may be an anti-CD3 antibody (e.g., OKT3, CRIS-7 or I2C) or an antigen-binding fragment of an anti-CD3 antibody. In some aspects, an agent that specifically binds CD3 is a single chain Fv fragment (scFv) of an anti-CD3 antibody. In some embodiments, the T-cell activation or co-stimulation molecule is selected from the group consisting of an anti-CD3 antibody, a ligand for CD28 (e.g., CD28L), and 41bb ligand (41BBL or CD137L). CD86, also known as B7-2, is a ligand for both CD28 and CTLA-4. In some embodiments, the ligand for CD28 is CD86. CD80 is an additional ligand for CD28. In some embodiments, the ligand for CD28 is CD80. In some embodiments, the ligand for CD28 is an anti-CD28 antibody or an anti-CD28 scFv fused to a transmembrane domain for display on the surface of the lentiviral particle. Lentiviral particles comprising one or more a T-cell activation or co-stimulation molecules made by engineering the packaging cell line may be made by methods provided by WO 2016/139463; however, the lentiviral vector systems disclosed therein perform the surface engineering using stable cell lines rather than a multicistronic vector, they do not link the envelope glycoprotein to the T-cell activation or co-stimulation molecules via linkers (e.g., 2A peptides), and they do not pseudotype the surface-engineered lentiviral particle with COCV G protein, as presently disclosed. The vectors of the disclosure may be used in the lentiviral vectors and other compositions and methods described in WO 2018111834 A1, WO 2018148224 A1, WO 2019/200056 A1, WO 2019156795 A1, each of which is incorporated herein by reference in its entirety. The non-viral protein may be a cytokine. In some embodiments, the cytokine may be selected from the group consisting of IL-15, IL-7, and IL-2. Where the non-viral protein used is a soluble protein (such as an scFv or a cytokine) it may be tethered to the surface of the lentiviral particle by fusion to a transmembrane domain, such as the transmembrane domain of CD8. Alternatively, it may be indirectly tethered to the lentiviral particle by use of a transmembrane protein engineered to bind the soluble protein. Further inclusion of one or more cytoplasmic residues may increase the stability of the fusion protein.

### 3 Lentiviral Particles

In another aspect the present disclosure further provides surface-engineered lentiviral particles, wherein the lentiviral particle is pseudotyped by the fusion glycoprotein or functional variant thereof, and wherein the lentiviral particle displays on its surface each of the plurality of polypeptides. The lentiviral particles of the present disclosure can be made with the packaging cell lines of the present disclosure, or with another packaging cell line, or by co-transfection of cultured cells, e.g. HEK-293T cells, with the multicistronic vector and other necessary plasmids (e.g., transfer plasmid and packaging plasmid(s)). In some embodiments, due to the multicistronic vector, the lentiviral particle will comprise one or more T-cell activation or co-stimulation molecules, which molecules can be, without limitation, an anti-CD3 antibody of single-chain variable fragment (scFv) fused to a transmembrane domain, an CD28 ligand, or 41bb ligand. In some embodiments, the lentiviral particle comprises a Cocal virus G (COCVG) protein, an anti-CD3 scFv, a human CD86, and a human CD137L, or functional variants thereof. In some embodiments, some of the T-cell activation or co-stimulation molecules are provided by the multicistronic vector and others are provided by another plasmid or by the packaging cell line. The cultured cells could be HEK-293T cells. In some cases, the cultured cells are genetically modified to lack MHC class I expression, MHC class II expression, or expression of inhibitory checkpoint ligands, such as PD-L1 (a PD-1 ligand), or ligands for TIM3

### 4 Methods of Use

In another aspect, the disclosure provides methods of generating surface-engineered lentiviral particles, comprising providing a cell in a culture medium; and transfecting the cell with the multicistronic vector, a transfer plasmid, and a packaging plasmid, simultaneously or sequentially. After transfection, the cell expresses a surface-engineered lentiviral particle.

In some embodiments, the titer of surface-engineered lentiviral particle in the culture medium after transfection is at least about as high as the titer of a pseudotyped lentiviral particle produced by the same method using pMD2.G plasmid in place of the mutlicistronic vector. In some embodiments, the titer of surface-engineered lentiviral particle after transfection is at least about 1×10⁶, 1×10⁷, 2×10⁷, 4×10⁷, 6×10⁷, 8×10⁷, or 1×10⁸ IU/ml. In some embodiments, after the transfecting step, the method includes harvesting the lentiviral particle from the culture medium.

In another aspect, the disclosure provides methods for treating a subject suffering from cancer, including the step of administering a surface-engineered lentiviral particle of the disclosure to the subject, wherein the cancer is treated in the subject.

In some embodiments, the disclosure provides a surface-engineered lentiviral particle for use in therapy. In other embodiments, the disclosure provides a surface-engineered lentiviral particle for use in a method of treating a cancer. In further embodiments, the disclosure provides a surface-engineered lentiviral particle for use in the manufacture of a medicament for treating cancer.

In some embodiments, the cancer is a solid tumor, such as a melanoma, non-small cell lung cancer, or breast cancer. The methods of the present disclosure may include treating any cancer, including, without limitation, acute granulocytic leukemia, acute lymphocytic leukemia, acute myelogenous leukemia, adenocarcinoma, adenosarcoma, adrenal cancer, adrenocortical carcinoma, anal cancer, anaplastic astrocytoma, angiosarcoma, appendix cancer, astrocytoma, basal cell carcinoma, B-cell lymphoma, bile duct cancer, bladder cancer, bone cancer, bone marrow cancer, bowel cancer, brain cancer, brain stem glioma, brain tumor, breast cancer, carcinoid tumors, cervical cancer, cholangiocarcinoma, chondrosarcoma, chronic lymphocytic leukemia, chronic myelogenous leukemia, colon cancer, colorectal cancer, craniopharyngioma, cutaneous lymphoma, cutaneous melanoma, diffuse astrocytoma, ductal carcinoma in situ, endometrial cancer, ependymoma, epithelioid sarcoma, esophageal cancer, Ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors, general, germ cell tumor, gestational trophoblastic disease, glioblastoma multiforme, glioma, hairy cell leukemia, head and neck cancer, hemangioendothelioma, Hodgkin lymphoma, Hodgkin lymphoma, Hodgkin's disease, hypopharyngeal cancer, infiltrating ductal carcinoma, infiltrating lobular carcinoma, inflammatory breast cancer, intestinal cancer, intrahepatic bile duct cancer, invasive/infiltrating breast cancer, islet cell cancer, jaw cancer, kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma, metastatic breast cancer, metastatic melanoma, metastatic squamous neck cancer, mixed gliomas, mouth cancer, mucinous carcinoma, mucosal melanoma, multiple myeloma, mycosis fungoides, myelodysplastic syndrome, nasal cavity cancer, nasopharyngeal cancer, neck cancer, neuroblastoma, neuroendocrine tumors, non-Hodgkin lymphoma, non-small cell lung cancer, oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer, ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary tumors, primary central nervous system, prostate cancer, rectal cancer, renal cell carcinoma, renal pelvis cancer, rhabdomyosarcoma, salivary gland cancer, sarcoma, bone sarcoma, soft-tissue sarcoma, uterine, sinus cancer, skin cancer, small cell lung cancer, small intestine cancer, soft tissue sarcoma, spinal cancer, spinal column cancer, spinal cord cancer, spinal tumor, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, triple-negative breast cancer, tubal cancer, tubular carcinoma, undiagnosed cancer, ureteral cancer, uterine adenocarcinoma, uterine cancer, uterine sarcoma, vaginal cancer, vulvar cancer.

In another aspect, the disclosure provides a method for expanding T-cells capable of recognizing and killing tumor cells in a subject in need thereof, including the steps of administering a lentiviral particle of the disclosure to the subject, such that T-cells capable of recognizing and killing tumor cells in the subject are transduced by the lentiviral particle and expanded. In some embodiments, the lentiviral particle is administered by intravenous injection or by intratumoral injection.

In some embodiments, the lentiviral particle comprises a targeting agent or the nucleic acid vector encodes a targeting agent. Exemplary targeting agents include antibodies and chimeric antigen receptors (“CAR”). The term “antibody” refers to an intact antigen-binding immunoglobulin of any kind, or a fragment thereof that itself specifically binds to the antibody's target antigen, and includes, for example, chimeric, humanized, fully human, and bispecific antibodies. The CAR used in the present disclosure in some embodiments comprises a binding domain which is specific for B-cells, e.g., specific for a CD-marker that can be found on B-cell lymphoma such as CD19, CD22, CD20 or CD79a, or CD19. T-cells that have been genetically engineered to express a CAR (e.g., a T-cell CAR) are exemplified in WO2007/131092. The targeting agent serves, in some embodiments, to direct cell-mediated immunity towards particular cell types, such as tumor cells.

In certain embodiments, a subject treated by the methods described herein may be a mammal. In some embodiments, a subject is a human, a non-human primate, a pig, a horse, a cow, a dog, a cat, a rabbit, a mouse or a rat. A subject may be a human female or a human male.

Combination therapies are also contemplated by the invention. Combination as used herein includes simultaneous treatment or sequential treatment. Combinations of methods of the invention with standard medical treatments (e.g., corticosteroids) are specifically contemplated, as are combinations with novel therapies. In some embodiments, a subject may be treated with a steroid (e.g. prednisone, prednisolone, deflazacort) to prevent or to reduce an immune response to administration of a lentiviral particle described herein. In certain cases, a subject may receive apheresis or an immune modulator if the subject expresses antibodies to the lentiviral particle described herein. In some embodiments, such immune modulators may be unnecessary, particularly when an immunosuppressive agent (e.g. tacrolimus or sirolimus) is administered. In some embodiments, rituxan is administered simultaneous to or sequential with treatment with a lentiviral particles. Rituxan may in some embodiments serve to block immune responses against the lentiviral particles

The lentiviral particles of the present disclosure may be administered by any route, including oral, nasal, intravenous, intra-arterial, intramuscularly, or intraperitoneal routes. In some embodiments, the lentiviral particle is administered by intravenous injection or by intratumoral injection. In some embodiments, the methods include administration of an immunosuppressive agent. In some embodiments, the immunosuppressive agent is administered by intravenous injection or administered orally. In some embodiments, the immunosuppressive agent is rapamycin, optionally administered at concentrations sufficient to maintain serum concentrations of rapamycin greater than 0.1 nM, 1 nM, or 10 nM. In some embodiments, the immunosuppressive agent is a rapalog, optionally administered at concentrations sufficient to maintain serum concentrations of the rapalog greater than 0.1 nM, 1 nM, or 10 nM.

In some embodiments, the immunosuppressive drug is administered simultaneously with the lentiviral particle; or the immunosuppressive drug is administered about 30 minutes, about 1 hour, about 2 hours, about 4 hours, about 5 hours or about 10 hours before or after the lentiviral particle is administered. In some embodiments, the immunosuppressive agent is tacrolimus, optionally administered at concentrations sufficient to maintain serum concentrations of tacrolimus greater than 0.1 nM, 1 nM, or 10 nM. In some embodiments, the immunosuppressive agent is cyclosporine, optionally administered at concentrations sufficient to maintain serum concentrations of cyclosporine greater than 0.1 nM, 1 nM, or 10 nM. In some embodiments, the immunosuppressive agent is an immunosuppressive drug. In some embodiments, the immunosuppressive agent is an immunosuppressive drug and the lentiviral vector comprises a nucleic acid sequence encoding a protein that provides resistance to said immunosuppressive drug

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating the lentiviral particle in the required amount in the appropriate solvent with various other ingredients enumerated above, as required. The injectable solutions may be prepared aspecticly or filter-sterilized.

### 5 Definitions

Unless defined otherwise, all technical and scientific terms used herein have the same meaning as commonly understood by those of ordinary skill in the art to which the invention belongs. For the purposes of the present invention, the following terms are defined below.

As used herein, “293T control particles” or “Mock (293T vector)” refer to lentiviral particles generated by transduction of 293T cells with a lentiviral vector. As used herein, “stimbeads” refers to a bead-based reagent used to stimulate T-cells during transduction. The label “+Control (vector+stimbeads)” refers transduction with 293T control particles with stimbeads.

As used herein, the term “HATSE cells” or “HATSE cell line” or “HATSE-293” refers a packaging cell line created by transducing 293T cells with lentiviral vector(s) encoding CD86 and CD137L and subjected to fluorescence-activated cell sorting (FACS) for cells that highly expressed both CD86 and CD137L one or more times. The label “(1× sorted) HATSE cell vector” refers to lentiviral particles generated by transduction of HATSE cells with a lentiviral vector after the HATSE cells are FACS-sorted for CD86⁺/CD173L⁺ double-positive cells one time. The label “(2× sorted)HATSE cell vector refers to lentiviral particles generated by transduction of HATSE cells with a lentiviral vector after the HATSE cells are FACS-sorted for CD86⁺/CD173L⁺ double-positive cells one time.

The articles “a,” “an,” and “the” are used herein to refer to one or to more than one (i.e. to at least one) of the grammatical object of the article. By way of example, “an element” means one element or more than one element.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

Any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%.

Throughout this specification, unless the context requires otherwise, the words “comprise”, “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure, or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “isolated” means material that is substantially or essentially free from components that normally accompany it in its native state. In particular embodiments, the term “obtained” or “derived” is used synonymously with isolated.

A “subject,” “patient” or “individual” as used herein, includes any animal that exhibits pain that can be treated with the vectors, compositions, and methods contemplated herein. Suitable subjects (e.g., patients) include laboratory animals (such as mouse, rat, rabbit, or guinea pig), farm animals, and domestic animals or pets (such as a cat or dog). Non-human primates and, preferably, human patients, are included.

As used herein “treatment” or “treating,” includes any beneficial or desirable effect associated with treatment. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of the disease or disorder. As used herein, “prevention” and similar words also includes reducing the intensity, effect, symptoms and/or burden of the disease or disorder prior to onset or recurrence.

As used herein, “therapeutically effective amount” or “an amount effective” or “effective amount” of a virus or lentiviral particle refers to the amount of the virus or lentiviral particle required to achieve a beneficial or desired prophylactic or therapeutic result, including clinical results.

A “prophylactically effective amount” refers to an amount of a virus or lentiviral particle effective to achieve the desired prophylactic result. Typically but not necessarily, since a prophylactic dose is used in subjects prior to or at an earlier stage of disease, the prophylactically effective amount is less than the therapeutically effective amount.

A “therapeutically effective amount” of a vector lentiviral particle may vary according to factors such as the disease state, age, sex, and weight of the individual, and the ability of the stem and progenitor cells to elicit a desired response in the individual. A therapeutically effective amount is also one in which any toxic or detrimental effects of the virus are outweighed by the therapeutically beneficial effects. The term “therapeutically effective amount” includes an amount that is effective to “treat” a subject (e.g., a patient).

An “increased” or “enhanced” amount of a physiological response, e.g., cellular activity or anti-tumor activity, is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the level of activity in an untreated subject.

A “decrease” or “reduced” amount of a physiological response is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the level of activity in an untreated cell or subject.

By “maintain,” or “preserve,” or “maintenance,” or “no change,” or “no substantial change,” or “no substantial decrease” refers generally to a physiological response that is comparable to a response caused by either vehicle, or a control molecule/composition. A comparable response is one that is not significantly different or measurably different from the reference response.

“Receptor-ligand binding,” “ligand binding,” and “binding” are used interchangeably herein to mean physical interaction between a receptor and a ligand or a synthetic ligand. Ligand binding can be measured by a variety of methods known in the art (e.g., detection of association with a radioactively labeled ligand).

As used herein, the terms “specific binding affinity” or “specifically binds” or “specifically bound” or “specific binding” are used interchangeably throughout the specification and claims and refer to that binding which occurs between a paired species of molecules, e.g., receptor and ligand. When the interaction of the two species produces a non-covalently bound complex, the binding which occurs is typically electrostatic, hydrogen-bonding, or the result of lipophilic interactions. In various embodiments, the specific binding between one or more species is direct. In one embodiment, the affinity of specific binding is about 2 times greater than background binding (non-specific binding), about 5 times greater than background binding, about 10 times greater than background binding, about 20 times greater than background binding, about 50 times greater than background binding, about 100 times greater than background binding, or about 1000 times greater than background binding or more.

In general, “sequence identity” or “sequence homology” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 80% to 100% and integer values therebetween. Typically, the percent identities between a disclosed sequence and a claimed sequence are at least 80%, at least 85%, at least 90%, at least 95%, or at least 98%.

The term “exogenous” is used herein to refer to any molecule, including nucleic acids, protein or peptides, small molecular compounds, and the like that originate from outside the organism. In contrast, the term “endogenous” refers to any molecule that originates from inside the organism (i.e., naturally produced by the organism).

The term “MOI” is used herein to refer to multiplicity of infection, which is the ratio of agents (e.g. viral particles) to infection targets (e.g. cells).

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

The section headings used herein are for organizational purposes only and are not to be construed as limiting the subject matter described.

The invention is further described in the following Examples, which do not limit the scope of the invention described in the claims.

### EXAMPLES

### Example 1: Lentiviral Particles Using Multicistronic Vector

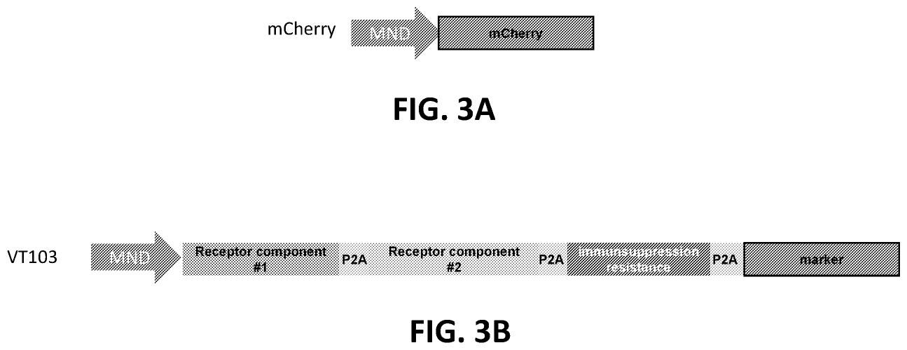

Envelope plasmids encoding CMV.VSVG (pMD2.8), CMV.Cocal, or MND.Cocal were generated. In each case the fusion glycoprotein (G protein) of the virus, either Vesicular stomatitis Indiana virus (VSV) or Cocal virus, was placed under the control of a strong promoter, either the CMV promoter or the MND promoter. As the transfer plasmids for these experiments, either a conventional mCherry-expressing transfer plasmid or a transfer plasmid comprising the fluorescence marker mCherry under the control of an MND promoter was used in some experiment. To model transfer plasmids with larger inserts, the transfer plasmid “VT103” shown in FIG. 3B was used.

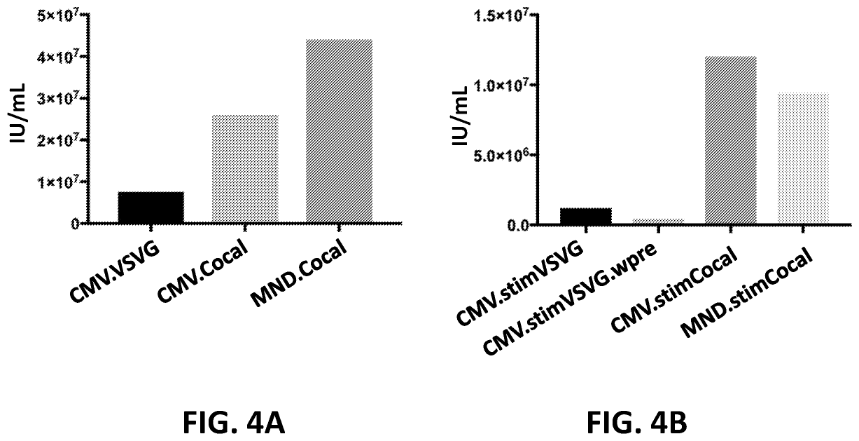

Titers of about 0.8×10⁷ to 4.5×10⁷ infectious units per milliliter (IU/ml) were achieved using the industry standard pMD2.G envelope plasmid expressing the VSV protein G under the control of a CMV promoter or envelope plasmids expressing Cocal virus protein G (COVG) under the control of a CMV promoter or under the control of an MND promoter (FIG. 4A). The transfer plasmid used was the pRRL-based plasmid pVT-103 and the packaging plasmids were pRSV-Rev and pMDLG-pRRE.

Next, the envelope plasmids were engineered to include a single non-viral gene before the G protein link by a 2A peptide sequence. Titers achieved with VSVG were about 0.2×10⁶ IU/mL or less (FIG. 4B). Titers of Cocal G-pseudotyped lentiviral particles were about 1×10⁷ or higher (FIG. 4B).

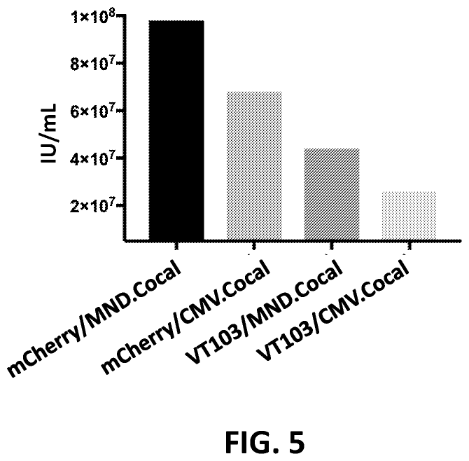

FIG. 5 shows comparative results with the mCherry or VT103 plasmids. The VT103 transfer plasmid (a large-insert, therapeutically relevant transfer plasmid) resulted in decreased packaging efficiency compared to mCherry alone. This demonstrates that experiments using conventional model transfer plasmids (e.g. mCherry or GFP alone) may in some cases show efficiencies higher than are achieved using a payload large enough to deliver a more desirable therapeutic construct, such as VT103, to the host cell.

Envelope plasmids encoding CD86-2A-anti-CD3 scFv-2A-CD137L-2A-COCVG (termed “h3stim.Cocal” or “h3stimCocal”) operatively linked to a CMV promoter or an MND promoter, as shown in FIGS. 6A-6B, were constructed and achieved efficiencies of at least about 1×10⁷ IU/mL (FIG. 6C).

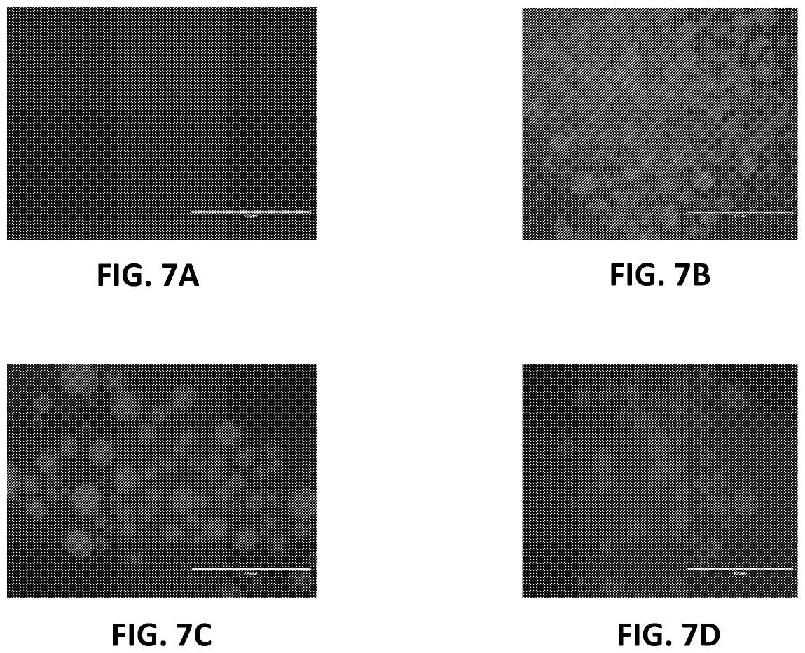

FIGS. 7A-7D demonstrate that lentiviral particles surface-engineered with a multicistronic vector encoding CD86-2A-anti-CD3 scFv-2A-CD137L-2A-COCVG activate target cells (shown by formation of clumps of cells in FIGS. 7C and 7D). For comparison, infection of cells with COVG-pseudotyped lentiviral particles without surface engineering is shown in FIGS. 7A-7B. Without anti-CD3/anti-CD28 stimulation, cells are not activated by COVG-psuedotyped lentiviral particles (FIG. 7A). Cells can be activated when these control lentiviral particles are supplemented by addition of anti-CD3-anti-CD28 dynabeads (FIG. 7B). Lenitiviral particles surface-engineered with the CD86-2A-anti-CD3scFv-2A-CD137L-2A-COCVG vector, using either the mCherry transfer plasmid (FIG. 7C) or VT103 (FIG. 7D), activate target cells even without dynabeads.

All of the features described herein (including any accompanying claims, abstract and drawings), and/or all of the steps of any method so disclosed, may be combined with any of the above aspects in any combination, except combinations where at least some of such features and/or steps are mutually exclusive. Specifically, any of the vectors described herein can be used in any of the described methods of treatment. Any and all such combinations are explicitly envisaged as forming part of the invention.

Those skilled in the art will recognize, or be able to ascertain using no more than routine experimentation, many equivalents to the specific embodiments of the invention described herein. Such equivalents are intended to be encompassed by the following claims.

What is claimed is:

1. A multicistronic vector for surface-engineering lentiviral particles, comprising a polynucleotide operatively linked to a promoter,
wherein the polynucleotide encodes a plurality of polypeptides joined by linkers comprising peptides capable of inducing ribosome skipping or self-cleavage, and
wherein the plurality of polypeptides comprise a fusion glycoprotein or functional variant thereof and one or more non-viral proteins capable of viral surface display.

2. The multicistronic vector of claim 1, wherein the linkers comprise 2A peptides each independently selected from the group consisting of P2A (SEQ ID NO: 14), T2A (SEQ ID NO: 15), E2A (SEQ ID NO: 16), and F2A (SEQ ID NO: 17).

3. The multicistronic vector of claim 1 or claim 2, where one or more of the linkers comprises a sequence encoding the residues Gly-Ser-Gly.

4. The multicistronic vector of any one of claims 1 to 3, wherein the the plurality of polypeptides comprises 2, 3, 4, 5, 6, 7, 8, 9, 10, 11, 12, 13, 14, 15, 16, 17, 18, 19 or 20 proteins capable of viral surface display.

5. The multicistronic vector of any one of claims 1 to 4, wherein the fusion glycoprotein or functional variant thereof is a viral fusion glycoprotein or a functional variant thereof.

6. The multicistronic vector of claim 5, wherein the viral fusion glycoprotein or functional variant thereof is Cocal virus G (COCVG) protein or a functional variant thereof.

7. The multicistronic vector of any one of claims 1 to 6, wherein the non-viral proteins capable of viral surface display comprise one or more non-viral proteins selected from a transmembrane-domain fusion of a single chain variable fragment (scFv) specific for human CD3 (anti-CD3 scFv), human CD86, and human CD137L, or functional variants thereof.

8. The multicistronic vector of claim 7, wherein:
a) the viral fusion glycoprotein is Cocal virus G (COCVG) protein or a functional variant thereof; and
b) the plurality of polypeptides comprises the anti-CD3 scFv, the human CD86, and human CD137L, or functional variants thereof.

9. The multicistronic vector of claim 8, wherein the plurality of polypeptides consists of the Cocal virus G (COCVG) protein, the anti-CD3 scFv, the human CD86, and the human CD137L, or functional variants thereof.

10. The multicistronic vector of claim 9, wherein the polynucleotide encodes one of:
a) COCVG-2A-anti-CD3scFv-2A-CD86-2A-CD137L;
b) anti-CD3 scFv-2A-COCVG-2A-CD86-2A-CD137L;
c) anti-CD3 scFv-2A-CD86-2A-COCVG-2A-CD137L;
d) anti-CD3 scFv-2A-CD86-2A-CD137L-2A-COCVG;
e) COCVG-2A-CD86-2A-anti-CD3 scFv-2A-CD137L;
f) CD137L-2A-anti-CD3 scFv-2A-COCVG-2A-CD86;
g) anti-CD3scFv-2A-CD137L-2A-COCVG-2A-CD86;
h) CD86-2A-anti-CD3 scFv-2A-COCVG-2A-CD137L;
i) CD86-2A-COCVG-2A-anti-CD3 scFv-2A-CD137L;
j) CD86-2A-COCVG-2A-CD137L-2A-anti-CD3 scFv;
k) CD86-2A-anti-CD3 scFv-2A-CD137L-2A-COCVG;
l) CD86-2A-CD137L-2A-anti-CD3scFv-2A-COCVG; and
m) CD137L-2A-anti-CD3 scFv-2A-CD86-2A-COCVG;

11. The multicistronic vector of claim 9, wherein the polynucleotide encodes CD86-2A-anti-CD3scFv-2A-CD137L-2A-COCVG.

12. The multicistronic vector of any one of claims 1 to 11, wherein the multicistronic vector is a lentiviral envelope plasmid capable of generating a lentiviral particle pseudotyped for COCV when co-transfected with a transfer plasmid and a packaging plasmid into a packaging cell line.

13. A cell comprising the multicistronic vector of any one of claims 1 to 12.

14. The cell of claim 13, wherein the cell comprises no other polynucleotides encoding any of the plurality of polypeptides encoded by the multicistronic vector other than the multicistronic vector itself.

15. A surface-engineered lentiviral particle produced by the cell of claim 13.

16. The surface-engineered lenviral particle of claim 15, wherein the lentiviral particle is pseudotyped by the fusion glycoprotein or functional variant thereof.

17. The surface-engineered lenviral particle of claim 15 or claim 16, wherein the lentiviral particle displays on its surface each of the plurality of polypeptides.

18. A pharmaceutical composition comprising the surface-engineered lenviral particle of any one of claims 15 to 17.

19. The use of a lentiviral particle produced by the cell of claim 13, the surface-engineered lentiviral particle of any one of claims 15 to 17, or the pharmaceutical composition of claim 18 as a medicament for the treatment of cancer.

20. A method of generating surface-engineered lentiviral particles, comprising:
a) providing a cell in a culture medium; and
b) transfecting the cell with the multicistronic vector of any one of claims 1 to 12, a transfer plasmid, and a packaging plasmid, simultaneously or sequentially;
whereby the cell expresses a surface-engineered lentiviral particle.

21. The method of claim 20, wherein the titer of surface-engineered lentiviral particle in the culture medium after step b) is at least about as high as the titer of a pseudotyped lentiviral particle produced by the same method using a pMD2.G plasmid in place of the multicistronic vector.

22. The method of claim 20 or claim 21, wherein the titer of surface-engineered lentiviral particle after step b) is at least about 1×10⁶, 1×10⁷, 2×10⁷, 4×10⁷, 6×10⁷, 8×10⁷, or 1×10⁸ IU/ml.

23. The method of any one of claims 20 to 22, comprising harvesting the lentiviral particle from the culture medium.

24. A method for treating a subject suffering from cancer, comprising:
administering the surface-engineered lentiviral particle of any one of claims 15 to 17 or the pharmaceutical composition of claim 18 to the subject,
whereby the cancer is treated in the subject.

25. A method for expanding T-cells capable of recognizing and killing tumor cells in a subject in need thereof, comprising:
administering the lentiviral particle of any one of claims 15 to 17 or the pharmaceutical composition of claim 18 to the subject,
whereby T-cells capable of recognizing and killing tumor cells in the subject are transduced by the lentiviral particle and expanded.

26. The method of claim 25, wherein the lentiviral particle is administered by intravenous injection.

27. The method of claim 25, wherein the lentiviral particle is administered by intratumoral injection.
